(19) United States
(12) Patent Application Publication
(10) Pub. No.: US 2022/0087813 A1
(43) Pub. Date: Mar. 24, 2022

(54) ANTI-PULFRICH MONOVISION OPHTHALMIC CORRECTION

(71) Applicants: THE TRUSTEES OF THE UNIVERSITY OF PENNSYLVANIA, Philadelphia, PA (US); CONSEJO SUPERIOR DE INVESTIGACIONES CIENTIFICAS (CSIC), Madrid (ES)

(72) Inventors: Johannes Daniel BURGE, Philadelphia, PA (US); Carlos Dorronsoro DIAZ, Madrid (ES); Victor Rodriguez LOPEZ, Madrid (ES)

(21) Appl. No.: 17/425,144

(22) PCT Filed: Jan. 31, 2020
(86) PCT No.: PCT/US2020/016232

§ 371 Date: Jul. 22, 2021

Related U.S. Application Data
(60) Provisional application No. 62/799,468, filed on Jan. 31, 2019.

(51) Int. Cl. A61F 2/16; G02C 7/02; A61F 2/14

(52) U.S. Cl. CPC A61F 2/1621; A61F 2/1451; G02C 7/027

(57) ABSTRACT

Methods, systems, and ophthalmic devices are described for correcting a misperception of depth of a moving object. An example ophthalmic device may comprise a first lens having a first optical characteristic that increases a distance of a focal point of a first eye. The ophthalmic device may comprise a second lens having a second optical characteristic that decreases a distance of a focal point of a second eye. The second lens may have a third optical characteristic that reduces a misperception of a distance of an moving object.

### CROSS-REFERENCE TO RELATED APPLICATIONS

This application claims the benefit of U.S. Provisional Application No. 62/799,468 filed Jan. 31, 2019, which application is hereby incorporated by reference in its entirety for any and all purposes.

### STATEMENT OF GOVERNMENT INTEREST

This invention was made with government support under R01-EY028571 awarded by the National Institutes of Health. The government has certain rights in the invention.

### BACKGROUND

The Pulfrich effect (referred to as the Classic Pulfrich effect in this disclosure) is a stereo-motion phenomenon first reported nearly 100 years ago. When a target oscillating in the frontoparallel plane is viewed with unequal retinal illuminance in the two eyes (induced, for example, with neutral density filters), the target appears to follow an elliptical trajectory in depth. This well-known illusory phenomenon has also been reported with unequal contrast between images. The effect occurs because the image with lower illuminance, or contrast, is processed more slowly. The mismatch in the processing speed causes a neural disparity, which results in the illusory motion in depth.

### SUMMARY

Methods, systems, and ophthalmic devices are described for correcting a misperception of depth of a moving object. An example ophthalmic device may comprise a first lens having a first optical characteristic that increases a distance of a focal point of a first eye. The ophthalmic device may comprise a second lens having a second optical characteristic that decreases a distance of a focal point of a second eye. The second lens may have a third optical characteristic that reduces a misperception of a distance of a moving object.

An example method may comprise outputting a first representation of a moving object to a first eye of a user; outputting a second representation of the moving object to a second eye of a user; receiving data indicative of an adjustment to a characteristic of one or more of the first representation or the second representation; determining, based on the data indicative of the adjustment, a lens characteristic associated with reducing a misperception of distance of the moving object; and outputting data indicative of the lens characteristic.

Additional advantages will be set forth in part in the description which follows or may be learned by practice. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate embodiments and together with the description, serve to explain the principles of the methods and systems.

FIG. 1A shows the classic Pulfrich effect.

FIG. 1B shows the reverse Pulfrich effect.

FIG. 1C shows effective neural image positions in the left and right eye as a function of time for the Classic Pulfrich effect, no Pulfrich effect, and the Reverse Pulfrich effect.

FIG. 1D shows monovision correction.

FIG. 2A shows points of subjective equality (PSEs), expressed as interocular delay, as a function of interocular differences in focus error (bottom axis, white circles) or as a function of differences in retinal illuminance for one human observer (top axis, black squares).

FIG. 2B shows psychometric functions for five of the nine differential blur conditions in FIG. 2A.

FIG. 2C shows maximum average delay for four different human observers in monovision-like optical conditions (white bars; 1.0 D interocular focus difference) vs. darkening one eye with a neutral density filter (gray bars; +0.15 optical density).

FIG. 2D shows binocular stimulus.

FIG. 2E shows points of subjective equality (PSEs) for one observer, expressed as onscreen interocular delay relative to baseline.

FIG. 2F shows psychometric functions for seven of the reverse Pulfrich conditions in FIG. 2E.

FIG. 2G shows psychometric functions for seven of the thirteen tested differential blur conditions (top) and all five of the tested differential retinal illuminance conditions (bottom).

FIG. 2H shows points of subjective equality (PSEs), expressed as interocular delay, as a function of interocular differences in focus error (bottom axis, white circles) or as a function of differences in retinal illuminance for one human observer (top axis, gray squares).

FIG. 3A shows illusion size in meters as a function of speed for an object moving left to right at 5.0 m for different monovision corrections strengths (curves).

FIG. 3B shows distance of cross traffic moving from left to right will be overestimated when the left eye is focused far and the right eye is focused near.

FIG. 3C shows distance of left to right cross traffic will be underestimated when the left eye is focused near and the right eye is focused far.

FIG. 3D shows original stimuli were composed of adjacent black-white (top) or white-black (bottom) 0.25°×1.00° bars.

FIG. 3E shows high-pass or low-pass filtered stimuli.

FIG. 3F shows resulting interocular delays.

FIG. 3G shows effect sizes for each human observer in multiple conditions, obtained from the best-fit regression lines.

FIG. 4 shows eliminating the Reverse Pulfrich effect.

FIG. 5A shows blur circle diameter in meters from aperture and defocus.

Solid and dashed lines show how two different aperture sizes (A and A′) cause two difference blur circle sizes (b and b′) for the same focus error.

FIG. 5B shows blur circle diameter in visual angle.

FIG. 6A shows geometry predicting illusion size (\hat{d}-d) for rightward motion with a neutral density filter in front of the left eye.

FIG. 6B shows stereo-geometry predicting illusion size (e.g., blur circle diameter) for rightward motion with a blurring lens in front of the left eye.

FIG. 7A shows reverse, classic, and anti-Pulfrich effects.

FIG. 7B shows discrimination thresholds.

FIG. 8A shows interocular delays with high- and low-pass filtered stimuli for each human observer.

FIG. 8B shows proportion of original stimulus contrast after low-pass filtering vs. high-pass filtering (solid vs. dashed curves, respectively) as a function of total black-white (or white-black) bar width.

FIG. 8C shows low-pass and high-pass filters with a 2 cpd cutoff frequency.

FIG. 8D shows low-pass filtered stimulus, original stimulus, and high-pass filtered stimulus with matched luminance and contrast.

FIG. 8E shows horizontal intensity profiles of the stimuli in FIG. 8D.

FIG. 8F shows amplitude spectra of the horizontal intensity profiles in FIG. 8E.

FIG. 9A shows predicted perceived motion trajectory (bold curve), given target motion directly towards the observer (dashed line), with an interocular retinal illuminance difference.

FIG. 9B shows predicted perceived motion trajectory, given target motion directly towards the observer, with an interocular blur difference.

FIG. 10 shows interocular delays for real and virtual neutral density filters.

FIG. 11A shows a comparison of delay trial lenses and delay contact lenses.

FIG. 11B shows optical density difference, interocular focus difference, and onscreen interocular delay for contact lenses.

FIG. 12 shows the similarity of measured effects with blur differences induced by contact lenses (clinically relevant) and trial lenses (used in the original experiment).

FIG. 13 is a block diagram illustrating an example computing device.

### DETAILED DESCRIPTION OF ILLUSTRATIVE EMBODIMENTS

Monovision is a common ophthalmic correction for presbyopia: one eye is focused for far distances and the other eye for near distances. It is well-known that monovision reduces the precision of vision, such as stereo-depth perception (for example, someone with monovision correction would find it difficult to thread a needle), especially of static objects, while the effects on the perception of moving objects is lesser known/studied. This invention offers an improvement to existing monovision corrections, which is important because these misperceptions (e.g., of motion and depth) can affect visual tasks such as driving.

This invention shows that those misperceptions can be corrected with a neutral filter (e.g., neutral density filter) of the adequate optical density over one of the eyes. If we consider a monovision correction in which both lenses have equal transmittance, when there is a target at far distance and the left eye is focused for near, we get a Reverse Pulfrich effect because the processing of the left eye's image is speeded up due to the blur. However, if the illuminance of the retinal image on left eye is reduced (for example reducing the transmittance of the lens in the left eye), that eye's processing speed would be slowed down and the effect cancelled, resulting in accurate motion perception. The resulting Anti-Pulfrich monovision correction provides binocular vision free of depth misperceptions.

The first step of the procedure to achieve Anti-Pulfrich monovision is the measurement or estimation of the Classic Pulfrich effect and or the Reverse Pulfrich effect in the patient for a certain amount of monovision. Other conditionings as visual habits, driving needs or ocular dominance can be included as input. The second step is calculating the Anti-Pulfrich monovision correction: optical power and optical density (i.e. Transmittance) in each eye. Depending on the specific correction type used, Anti-Pulfrich monovision can be achieved by different means. Contact Lenses, Intraocular lenses and other ocular implants can be tinted. They can be ordered with the appropriate tint, retrieved from stock or even tinted on site. Laser refractive surgery and small aperture corrections can be combined with neutral filters in additional corrections (typically contact lenses or sunglasses) with asymmetric transmission between eyes. The invention discloses the concept of ‘Reverse Pulfrich effect’ and uses it to produce a new kind of ophthalmic corrections named ‘Anti-Pulfrich Monovision’. Disclosed herein are 1) Anti-Pulfrich Monovision corrections, 2) the procedures to prescribe them to patients and 3) the system/calculations used in the prescription.

During monovision corrections for presbyopia, each eye is fit with a lens that sharply focuses light from a different distance, causing differential image blur between the two eyes. Approximately 10 million people in the United States have a monovision correction, but little is known about how differential blur affects motion perception. We investigated by measuring the Pulfrich effect, a stereo-motion phenomenon first reported nearly 100 years ago.

When a moving target is viewed with unequal retinal illuminance or contrast in the two eyes, the target appears to be closer or further in depth than it actually is, depending on its frontoparallel direction. The effect occurs because the image with lower retinal illuminance or contrast is processed more slowly. The mismatch in processing speed causes a neural disparity, which results in the illusory motion in depth. What happens with differential blur? Remarkably, results show that differential blur causes a Reverse Pulfrich effect, an apparent paradox. Blur reduces contrast and should therefore cause processing delays. But the Reverse Pulfrich effect implies that the blurry image is processed more quickly. The paradox is resolved by recognizing i) that blur reduces the contrast of high-frequency image components more than low-frequency image components, and ii) that high spatial frequencies are processed more slowly than low spatial frequencies, all else equal. Thus, this new version of a 100-year-old illusion is explained by known properties of the early visual system. The illusion sizes are large enough to impact public safety.

Introduction

In the year 2020, 2.1 billion people in the world, and 120 million people in the United States of America, will have presbyopia. Presbyopia, a part of the natural aging process, is the loss of near focusing ability due to the stiffening of the crystalline lens inside the eye. All people develop presbyopia with age, so the number of affected people increases as the population ages. The first symptoms appear at approximately 40 years old. Presbyopia is fully developed at age 55. Without correction, presbyopia prevents people from reading and from effectively using a smartphone.

Many corrections exist for presbyopia. Reading glasses, bifocals, and progressive lenses are well known examples. Less well known are monovision corrections. With a monovision correction, each eye is fit with a lens that sharply focuses light from a different distance, providing ‘near vision’ in one eye and ‘far vision’ in the other. Monovision thus causes differential blur in the left- and right-eye images of a target at a given distance. For patients in which the correction is successful, the visual system preferentially processes the higher quality and suppresses the lower quality of the two images. The consequence is an increase in the effective depth of field without many of the drawbacks of other treatments (e.g. the seam in the visual field caused by bifocals). Unfortunately, monovision does not come without its own set of drawbacks. Monovision degrades stereoacuity and contrast sensitivity, deficits that hamper fine-scale depth discrimination and reading in low light. Monovision is also thought to cause difficulties in driving and has been implicated in an aviation accident. Despite these drawbacks, many people prefer monovision corrections to the other treatments for presbyopia.

Approximately 12.5 million people in the United States currently have a monovision correction (see Supplement). This number will increase in the coming years; the population is aging with the baby boomers and monovision is the most popular contact lens correction for presbyopia. A full understanding of the effects of monovision on visual perception is therefore critical, both for sound optometric and ophthalmologic practice and for the protection of public safety. Unfortunately, there is no literature on how the differential blur induced by monovision impacts motion perception.

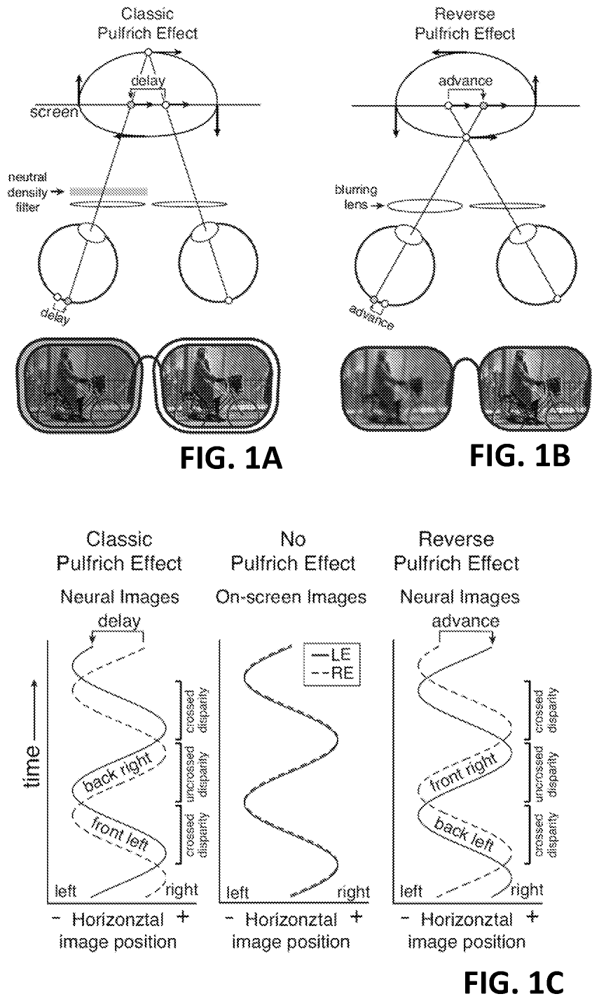

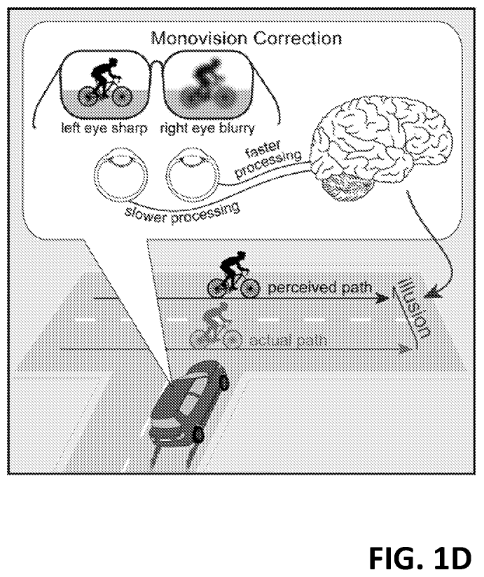

FIGS. 1A-B show the Classic and Reverse Pulfrich effects. FIG. 1A shows the classic Pulfrich effect. A neutral density filter in front of the left eye causes sinusoidal motion in the frontoparallel plane to be misperceived in depth (i.e. illusory clockwise motion from above: right in back, left in in front). The effect occurs because the response of the eye with lower retinal illuminance is delayed relative to the other eye, causing a neural disparity.

FIG. 1B shows the Reverse Pulfrich effect. A blurring lens in front of the left eye causes illusory motion in depth in the other direction (i.e. counter-clockwise from above: right in front, left in back). The effect occurs because the response of the eye with increased blur is advanced relative to the other eye, causing a neural disparity with the opposite sign.

FIG. 1C shows effective neural image positions in the left and right eye as a function of time for the Classic Pulfrich effect, no Pulfrich effect, and the Reverse Pulfrich effect.

Disclosed herein is an investigation of the impact of differential blur on motion perception by measuring the Pulfrich effect, a stereo-motion phenomenon that was first reported nearly 100 years ago. When a target oscillating in the frontoparallel plane is viewed with unequal retinal illuminance in the two eyes, the target appears to move along an elliptical trajectory in depth (FIG. 1A). The effect occurs because the visual system processes the image in the eye with lower retinal illuminance or contrast more slowly than the image in the other eye. The mismatch in processing speed causes a neural binocular disparity, a difference in the effective retinal locations of target images in the two eyes, which results in the illusory motion in depth.

The Pulfrich effect has been researched extensively since it was first discovered. The effect is elicited both by interocular luminance differences and by interocular contrast differences, and its magnitude is known to depend on overall luminance, dark adaptation, and numerous other factors. Around the turn of the century, a flurry of work debated what the effect reveals about the neural basis of stereo and motion processing. But again, it is not known whether the Pulfrich effect occurs under conditions similar to those induced by monovision corrections.

Do interocular blur differences, like interocular illuminance and contrast differences, cause misperceptions of motion? More specifically, does blur reduce processing speed and cause a Pulfrich effect? Disclosed herein are findings that rather than causing a Classic Pulfrich effect, differential blur causes a Reverse Pulfrich effect (e.g., as shown in FIG. 1B). In the Classic Pulfrich effect, decreasing retinal illuminance or contrast of the left eye, observers perceive ‘front left’ motion (i.e. clockwise motion when viewed from above). However, when the left eye is blurred, observers perceive ‘front right’ motion (i.e. counter-clockwise motion when viewed from above).

The discovery of the Reverse Pulfrich effect implies an apparent paradox. Blur reduces contrast and should therefore cause the blurry image to be processed more slowly, but the Reverse Pulfrich effect implies that the blurry image is processed more quickly than the sharp image (e.g., see FIG. 1C). At first, this finding appears at odds with large body of neurophysiological and behavioral results. Low contrast images are known to be processed more slowly at the level of early visual cortex and at the level of behavior.

The paradox may be resolved by recognizing two facts. First, blur reduces the contrast of high spatial frequency image components more than low-frequency image components. Second, extensive neurophysiological and behavioral literatures indicate that high spatial frequencies are processed more slowly than low spatial frequencies, all else equal. The explanation is likely that the blurry image is advanced in time relative to the sharp image because the high spatial frequency components in the sharp image decrease the speed at which the sharp image is processed. Thus, a new version of a 100-year-old illusion can be explained by known properties of the early visual system.

Results

To measure the impact of differential blur on the perception of motion trial lenses were used to induce interocular blur differences, and a haploscope for dichoptic presentation of moving targets (see Methods). The strength of the Pulfrich effect was measured using a one-interval two-alternative forced choice (2AFC) experiment. On each trial, a binocular target oscillated sinusoidally from left to right (or right to left) in front of the observer. The onscreen interocular delay of the target images was under experimenter control. If the onscreen interocular delay is zero, the stereoscopic target is specified by onscreen disparity to be moving in the plane of the screen. If the onscreen delay is non-zero, it specifies a stereoscopic target moving on an elliptical trajectory outside the plane of the monitor. The task was to report whether the target was moving leftward or rightward when it appeared to be closer than the screen (i.e. clockwise or counter-clockwise when viewed from above (e.g., see FIG. 1C). Human observers made these judgments easily and reliably.

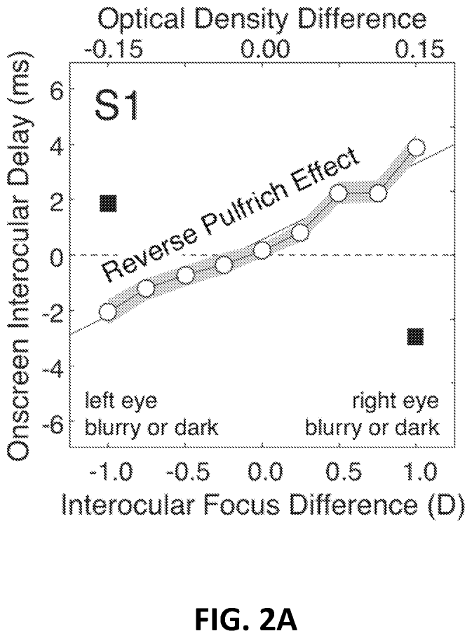

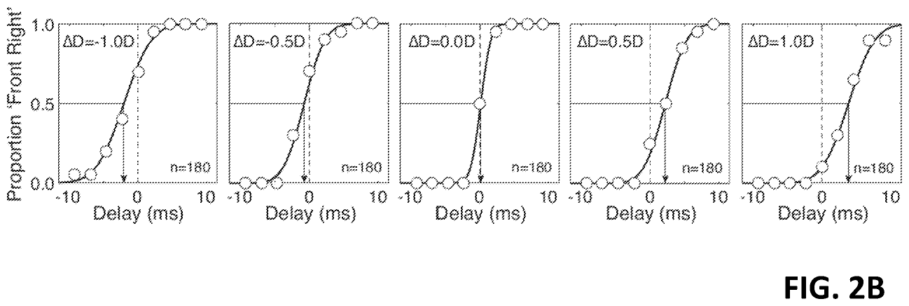

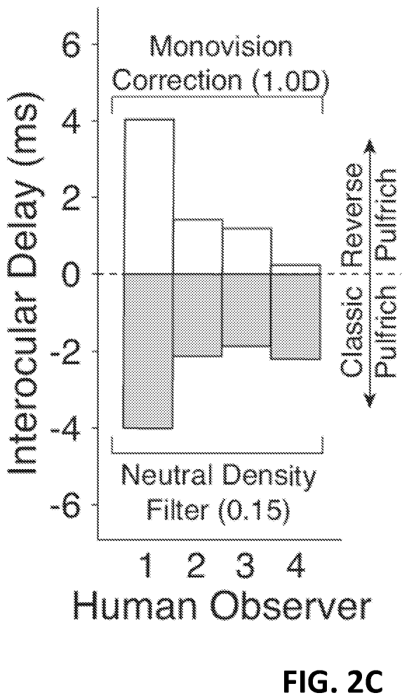

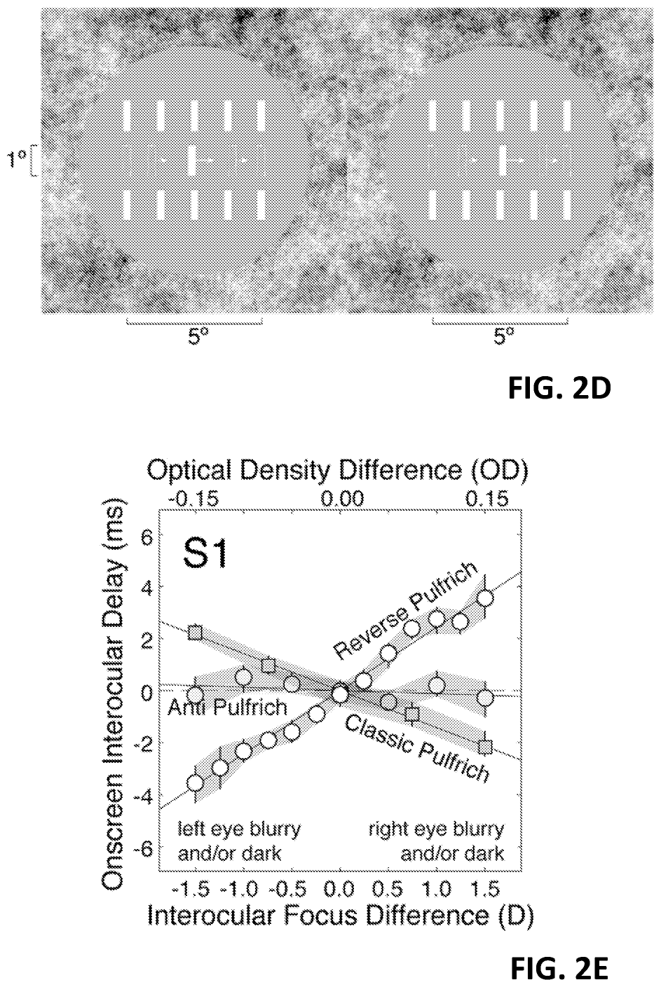

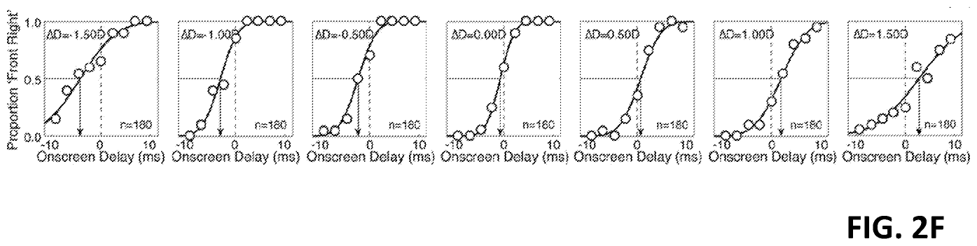

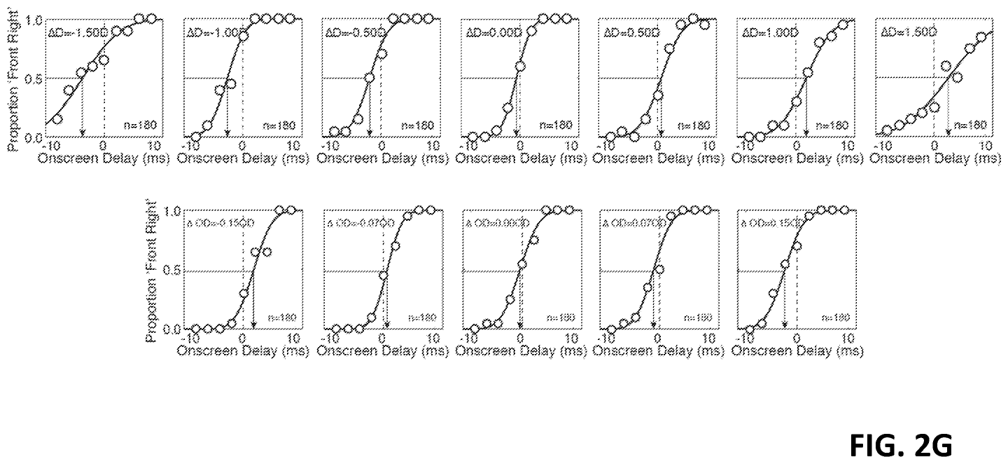

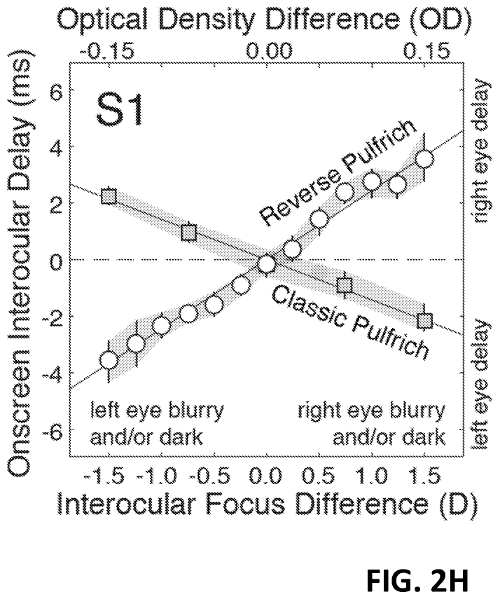

FIGS. 2A-C shows shows Reverse Pulfrich and Classic Pulfrich effects. FIG. 2A shows point of subjective equality (PSEs), expressed as interocular delay, as a function of interocular differences in focus error (bottom axis, white circles) or as a function of differences in retinal illuminance for one human observer (top axis, black squares). Differences in focus error were introduced by defocusing each eye from 0.0 D to 1.0 D, while keeping the other eye sharply focused. Differences in retinal illuminance were induced by placing neutral density filters in front of one eye, while leaving the other eye unfiltered (black squares). The best fit regression line is also shown. FIG. 2B shows Psychometric functions for five of the nine differential blur conditions in FIG. 2A. The arrows indicate the PSE for each condition. FIG. 2C shows maximum average delay for four different human observers in monovision-like optical conditions (white bars; 1.0 D interocular focus difference) vs. darkening one eye with a neutral density filter (gray bars; +0.15 optical density). Reverse and Classic Pulfrich effect sizes are correlated within observers (R=0.62; p<0.01).

For a given interocular difference in focus error, the proportion of times that the observers reported ‘front-right’ (i.e. counter-clockwise when viewed from above) were measured as a function of the onscreen interocular delay between the left and right eye images. Performance was summarized with the point of subjective equality (PSE), the 50% point on each psychometric function (e.g., as shown FIG. 2A). The PSE specifies the amount of onscreen interocular delay required to make the target appear to move in the plane of the display screen (i.e. no motion in depth).

The magnitude of the Reverse Pulfrich effect increases linearly with the difference in focus error (e.g., as shown in FIG. 2B). When the left-eye retinal image is blurry and the right-eye retinal image is sharp (negative interocular difference in focus error), the left-eye onscreen image must be delayed for the target to be perceived as moving in the plane of the screen (negative PSE shift). Conversely, when the right-eye image is blurry (positive interocular difference in focus error), the right-eye image must be delayed (positive PSE shift). These results indicate that the blurrier image is processed faster than the sharper image. For the first human observer, a +1.0 D difference in focus error led to an average interocular delay of approximately +2.75 ms (FIG. 2B).

The pattern characterizing the performance of the first human observer is consistent across all four human observers (e.g., as shown in FIG. 2C). The largest differences in focus error (i.e. +1.0 D) elicited interocular delays ranging from +0.25-2.75 ms across human observers (e.g., as shown in FIG. 2C, white bars). These differences in processing speed appear to be modest. However, as we will soon see, a few milliseconds difference in processing speed can lead to dramatic illusions in depth.

But first, to verify that the differential blur indeed causes a Reverse Pulfrich effect, we measured the Classic Pulfrich effect. To do so, we systematically reduced the retinal illuminance to one eye while leaving the other eye unperturbed (see Methods section below). As expected, the pattern of PSE shifts reverses (e.g., as shown in FIG. 2B, black squares and FIG. 2C, gray bars). When the left eye's retinal illuminance is reduced, the left eye onscreen image must be advanced in time for the target to be perceived as moving in the plane of the screen (positive PSE shift), and vice versa. These results indicate, consistent with classic findings, that the image with lower retinal illuminance is processed more slowly than the brighter image.

The interocular differences in processing speed vary across observers, but the magnitude of the changes due to blur and retinal illuminance differences are correlated for each observer (R=0.62; FIG. 2C). Future large-scale studies should measure the range and attempt to determine the origin of these inter-observer differences across the presbyopic population.

Discussion

Motion Illusions in the Real World

Monovision corrections cause misperceptions of motion. How large are these misperceptions likely to be in daily life? If the illusions are small, they will impose no impediment and can be safely ignored. If the illusions are large, they may pose a significant issue. One must be careful when generalizing laboratory results to the real world. Differences in viewing conditions can change the magnitude of effects. The same focus error causes less blur with smaller pupils, the same interocular difference in processing speed results in larger binocular disparities at faster speeds, and the same disparity specifies larger depths at longer viewing distances. Thus, all these factors—pupil size, target speed, and viewing distance—must be taken into account when predicting the severity of misperceptions that wearers of monovision corrections are likely to experience in daily life (see Methods).

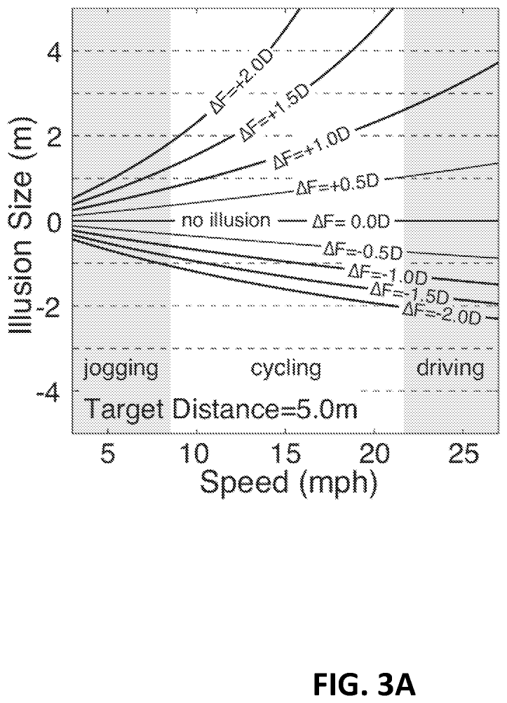

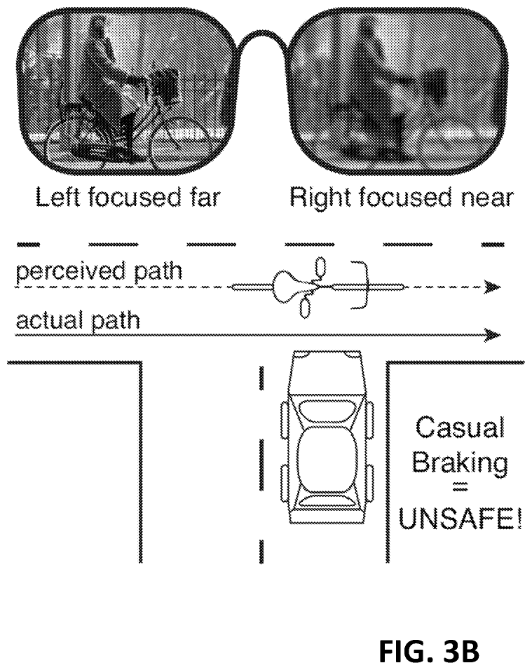

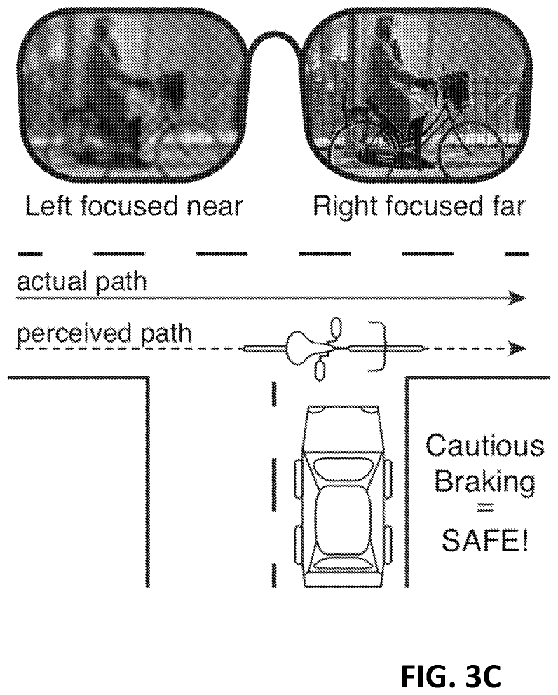

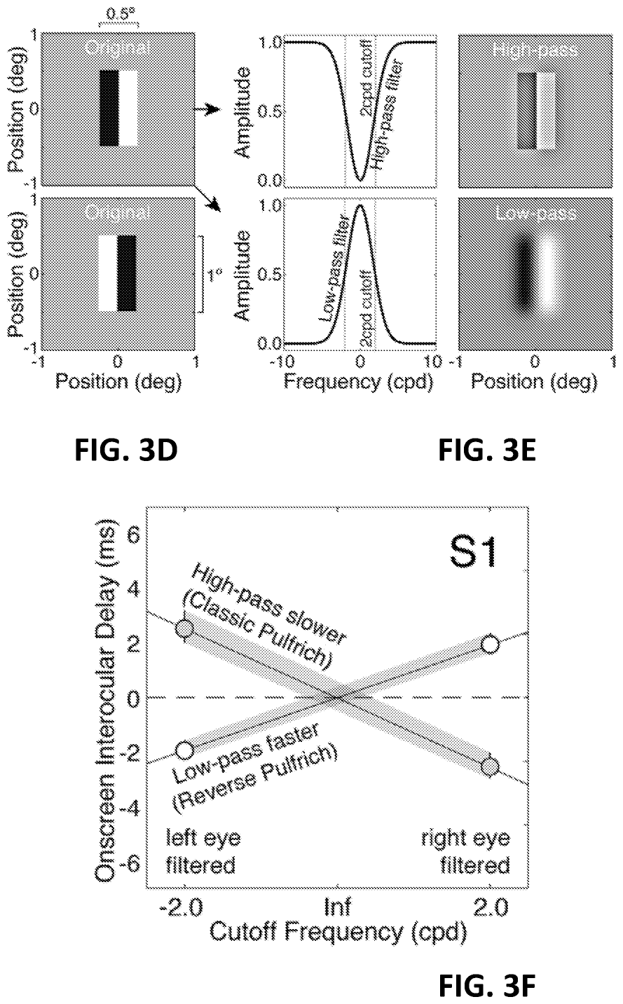

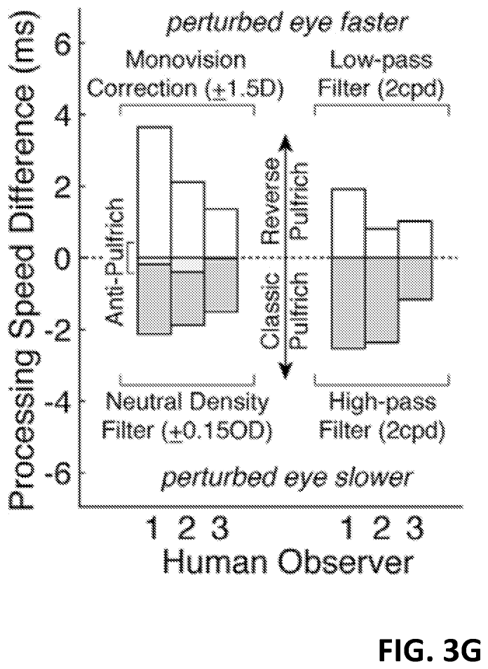

FIGS. 3A-B show monovision corrections and misperceptions of depth. FIG. 3A shows illusion size in meters as a function of speed for an object moving left to right at 5.0 m for different monovision corrections strengths (curves). Monovision correction strengths (e.g., interocular focus difference, ΔF; see Methods section below) typically range between 1.0 D and 2.0 D (thicker curves); strengths of 0.5 D are typically not prescribed, but we show them for completeness (thinner curves). Shaded regions show speeds associated with jogging, cycling, and driving. Illusion sizes are predicted directly from stereo-geometry (e.g., see Methods section below) assuming a pupil size (2.1 mm) that is typical for daylight conditions, and assuming interocular delays that were measured in the first human observer (e.g., as shown in FIG. 2A).

FIG. 3B shows the distance of cross traffic moving from left to right will be overestimated when the left eye is focused far (i.e. sharp image of cross traffic) and the right eye is focused near (i.e. blurry image of cross traffic). FIG. 3C shows distance of left to right cross traffic will be underestimated when the left eye is focused near and the right eye is focused far.

Consider a target object, five meters away, moving from left to right in daylight conditions. Illusion sizes, as predicted by stereo geometry, are shown (e.g., FIG. 3A) for one observer as a function of target speed with different monovision corrections strengths, which typically range between 1.0 D and 2.0 D. Consider the curve associated with a +1.5 D interocular difference in optical power (far lens over left eye), a common monovision correction strength. With this correction, the distance of a target at 5.0 meters moving from left to right at 15 miles per hour will be overestimated by 2.8 m (see Methods). This, remarkably, is the width of a narrow street lane. If the prescription is reversed (−1.5 D; far lens over right eye), target distance will be underestimated by 1.3 m. Stronger monovision corrections and faster speeds will increase the illusion sizes (see Supplement). Illusion sizes should also increase in dim light (e.g. driving at dawn, dusk or night); the differential blur associated with a given focus error will increase because of the accompanying increase in pupil size, and neural factors tend to exaggerate latency differences.

Illusions of this size will not only be disturbing for someone with a monovision correction, these illusions have the potential to endanger public health. In countries where motorists drive on the right side of the road (e.g., USA), cars and cyclists approaching in the near lane of cross traffic move from left to right. Placing the far lens in the left eye will cause distance overestimation, which may result in casual braking and increase the likelihood of traffic accidents (e.g., as shown in FIG. 3B). Placing the far lens in the right eye may be advisable. The resulting distance underestimation should result in more cautious braking and reduce the likelihood of collisions (e.g., as shown in FIG. 3C). In countries where motorists drive on the left side of the road (e.g., United Kingdom), the opposite practice should be considered (i.e. far lens in left eye). The current standard is to place the far lens in the dominant eye, but this does not appear to increase patient acceptance rate or patient satisfaction. Although the scenarios just discussed are not the only ones that should be considered, they may invite reexamination of standard optometric practice.

In the real world, many monocular cues exist that tend to indicate the correct rather than illusory depths. It will be of clinical and scientific interest to examine how the Reverse Pulfrich effect manifests in the rich visual environment of the real world. This issue could be addressed with virtual- or augmented-reality headsets that are now capable of providing nearphotorealistic computer-generated graphical renderings while at the same time allowing the precise programmatic control of the visual stimulus. Thus, although the literature on cue combination suggests the magnitude of the Reverse Pulfrich effect will be somewhat reduced from the stereo-geometric predictions in FIG. 3A, the predictions constitute a useful benchmark for future studies.

Eliminating Monovision-Induced Motion Illusions

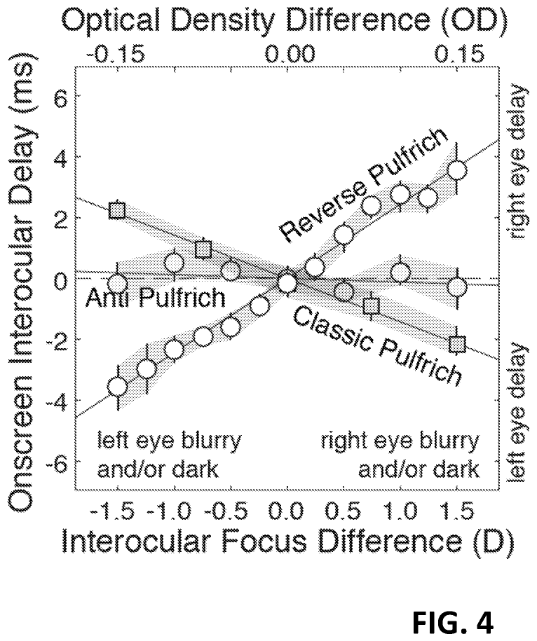

Reconsidering prescribing practices is one approach to minimizing the negative consequences of monovision-induced motion illusions, but it is clearly not the perfect solution. It would be far better to eliminate the illusions altogether. Increased blur and reduced retinal illuminance have opposite effects on processing speed, so it may be possible to null the two effects by tinting the lens that forms the blurry image (e.g., as illustrated in FIG. 4). FIG. 4 shows eliminating the Reverse Pulfrich effect. Appropriately tinting the blurring lens can eliminate the Reverse Pulfrich effect (shaded circles; see Methods section below). For reference, the data from FIGS. 2A-C with differential blur (white circles; Reverse Pulfrich effect) and differential retinal illuminance (shaded squares; Classic Pulfrich effect) is also shown.

Adaptation

Do motion illusions diminish or disappear as wearers adapt to monovision corrections over time? The question has never been asked with monovision corrections and the Reverse Pulfrich effect. However, if the literature on the Classic Pulfrich effect is any indication, the severity of motion illusions may increase until reaching an asymptote at steady state.

Methods

Observers: Four human observers ran in the experiment. All human observers had normal or corrected to normal visual acuity (20/40), and normal stereoacuity as confirmed by the Titmus Stereo Test.

Apparatus: Stimuli were displayed on a custom-built four-mirror haploscope. Left- and right-eye images were presented on two identical Vpixx Viewpixx LED monitors. Monitors were calibrated (i.e. the gamma functions were linearized) using custom software routines. The monitors had a size of 52.2×29.1 cm, spatial resolution of 1920×1080 pixels, a native refresh rate of 120 Hz, and a mean luminance of 100 cd/m2, which yields a pupil diameter of 2.5 mm.

The monitors were daisy-chained together and controlled by the same AMD FirePro D500 graphics card with 3 GB GDDR5 VRAM, to ensure that the left and right eye images were presented synchronously. Simultaneous measurements with two optical fibers connected to an oscilloscope confirmed that the left and right eye monitor refreshes occurred within 5 microseconds of one another. Custom firmware was written so that each monitor was driven by a single color channel; the red channel drove the left monitor and the green channel drove the right monitor. The single-channel drive to each monitor was then split to all three channels to enable gray scale presentation.

Human observers viewed the monitors through mirror cubes with 2.5 cm circular openings positioned one inter-ocular distance apart. Heads were stabilized with a chin and forehead rest. The haploscope mirrors were adjusted such that the vergence distance matched the optical distance of the monitors. The light path from monitor to eye was 100 cm, as confirmed both and by a laser ruler measurement and by a visual comparison with a real target at 100 cm. At the 100 cm viewing distance, each pixel subtended 1.09 arcmin. Anti-aliasing enabled sub-pixel resolution that permitted accurate presentations of disparities as small as 15-20 arcsec.

Stimuli

The stimulus was a binocularly presented 0.125×1.00° vertical bar. In each eye, the image of the bar moved left and right with a sinusoidal profile. An interocular phase shift between the left and right-eye images introduced a spatial disparity between the left- and right-eye bars. The left and right-eye bar positions onscreen were given by

x_{L}(t)=A cos(2πωt+ϕ₀+ϕ) (1a)

x_{R}(t)=A cos(2πωt+ϕ₀) (1b)

where x_{L} and x_{R} are the left and right eye x-positions in degrees of visual angle, A is the movement amplitude in degrees of visual angle, co is the temporal frequency, t is time, ϕ₀ is the starting phase which in our experiment determines whether the target starts on the left or the right side of the display, and ϕ is the phase shift (i.e. difference) between the images.

The interocular delay (i.e. temporal shift) in seconds associated with a particular phase shift is

Δt=ϕ/(2πω) (2)

Negative values indicate the left eye onscreen image is delayed relative to the right; positive values indicate the left eye onscreen image is advanced relative to the right.

When the inter-ocular phase shift equals zero, the virtual bar moves in the fronto-parallel plane at the distance of the monitors. When the interocular phase shift is non-zero, a spatial binocular disparity results, and the virtual bar follows a near-elliptical trajectory of motion in depth. The binocular disparity in radians of visual angle as a function of time is given by

$$δ ( t ) = {x}_{L} ( t ) - {x}_{R} ( t ) = - 2 Asin ( \frac{ϕ}{2} ) sin ( 2 πω t + {ϕ}_{0} + \frac{ϕ}{2} ) \qquad ( 3 )$$

The binocular disparity takes on its maximum magnitude when the perceived stimulus is directly in front of the observer and is moving at maximum speed. When the stimulus is moving to the right, the maximum disparity in visual angle is given by δₘₐₓ=2A sin(ϕ/2).

In our experiment, the movement amplitude was 2.5° of visual angle (i.e. 5.0° total change in visual angle in each direction), the temporal frequency was 1 cycle per second, and the starting phase ϕ₀ was randomly chosen to be either 0 or π. Restricting the starting phase to these two values meant that the stimuli started either 2.5° to the right or 2.5° to the left of center on each trial. The interocular phase shift ϕ ranged between ±200 arcmin at maximum, corresponding to interocular delays of ±9.3 ms and to maximum binocular disparities ±−8.7 arcmin at maximum. The range and particular values were adjusted to the sensitivity of each human observer.

Procedure

The observer's task was to report whether the stimulus appeared to move leftward or rightward when the stimulus was nearer to the observer in its virtual trajectory in depth. Using a one interval two-alternative forced choice procedure, nine-level psychometric functions were collected in each condition using the method of constant stimuli. Each function was fit with a cumulative Gaussian using maximum likelihood methods. The 50% point on the psychometric function—the point of subjective equality (PSE)—indicates the interocular delay (or equivalently, interocular phase shift) needed to null the interocular difference in processing speed. The pattern of PSEs is fit via linear regression (see below).

Defocus and Blur

The interocular focus difference is the magnitude of the focus error (i.e. defocus) in the right eye minus the magnitude of the focus error in the left eye

ΔF=|ΔDᵣ|−|ΔD_{L}| (4)

where ΔD=D_{focus}−D_{target} is the focus error, the difference between the dioptric distances of the focus and target points. To manipulate the amount of defocus blur in each eye, we positioned trial lenses ˜12 mm from each eye, centered on each optical axis, between each eye and the front of the mirror cubes of the haploscope.

Human observers ran in nine focus error conditions. We had four conditions with focus error in the left eye ranging from 0.25 D to 1.00 D in 0.25 D steps while the right eye was sharp (i.e. ΔF<0.0 D), one condition in which both eyes were sharp (i.e. ΔF=0.0 D), and four conditions with focus error in the right eye ranging from 0.25 D to 1.00 D in 0.25 D steps while the left eye was sharp (i.e. ΔF>0.0 D). Thus, each eye was defocused from 0.0 D to 1.0 D in 0.25 D steps while the other eye was kept sharp.

In the condition in which both eyes were sharply focused, the optical distances of the left- and right-eye monitors were set to optical infinity with +1.00 D trial lenses. When human observers fully relaxed the accommodative power of their eyes, the monitor was clearly focused. All observers indicated that they could sharply focus the monitor under these conditions. Also, because each trial lens absorbs a small fraction of the incident light, having a trial lens in front of each eye in all conditions ensures that retinal illuminance is matched in both eyes in all conditions. To induce interocular differences in focus error, we positioned a stronger positive lens (i.e. +1.25 D, +1.50 D, +1.75 D, +2.00 D) in front of one eye. This procedure places one eye's monitor beyond optical infinity, thereby introducing hyperopic focus errors that could not be cleared by accommodation. Before each run, the observer viewed a test target to confirm that he/she could clearly focus targets at optical infinity in the 0.0 D baseline condition.

One potential concern is that the eyes could accommodate independently to clear the blur in each eye, nulling our attempts to induce interocular differences. There are several reasons to think this was not in fact an issue in the current experiments. First, accommodation in the two eyes tends to be strongly coupled, especially for targets straight ahead at distances at or beyond 1.0 m. Thus, unequal optical powers in the two eyes should tend to induce differential blur. Second, focusing one eye beyond optical infinity (see above) minimizes the possibility that differential optical power could be nulled by differential accommodation in the two eyes. Third, the consistent pattern of results across conditions and observers suggest differential blur was successfully induced. Nevertheless, because accommodation was not paralyzed in the present experiments, it is still possible that some aniso-accommodation occurred. Some fraction of the inter-observer variability in the size of the Reverse Pulfrich effect could be due to different amounts of aniso accommodation amongst the observer population; this could be studied in the future both by measuring accommodative state during the experiments and/or by paralyzing accommodation. However, it is also possible (and we think more probable) that the inter-observer variability in effect size is due to neural factors. This may be because the size of the Reverse Pulfrich effect predicts the size of the Classic Pulfrich effect in each individual observer (e.g., see FIG. 2C).

Neutral Density Filters

To induce interocular differences in retinal illuminance ‘virtual’ neutral density filters were place in front of the eyes. To do so, the optical density of the virtual filter was converted to transmittance, the proportion of incident light that is passed through the filter, using the standard expression T=10^{-0D} where T is the transmittance and OD is the optical density. Then, the luminance of one eye's monitor was reduced by a scale factor equal to the transmittance. To verify that the calculations and monitor calibrations were accurate, we compared human performance with virtual and real neutral density filters on all observers for an optical density of 0.15, corresponding to a transmittance of 70.8%. In all observers, performance with the real and virtual neutral density filters is essentially identical.

The interocular difference in optical density ΔO=OD_{R}−OD_{L} is the difference between the optical density of filters placed over the right and left eyes. Human observers ran in five conditions with virtual neutral density filters, with equally spaced interocular differences in optical density between −0.15 and 0.15. We had two conditions with a filter in front of the left eye (i.e. ΔO<0.00), one condition in which both eyes were unfiltered (i.e. ΔO=0.00), and two conditions with a filter in front of the right eye (i.e. ΔO>0.00).

Generalizing Laboratory Results to the Real World

To make predictions for how monovision corrections will cause misperception of motion in the real world, it is important to take into account the differences in viewing conditions that may impact the magnitude of the effects. The conditions were set by differences in focus error, but the Reverse Pulfrich effect must actually be mediated by differences in image blur. The amount of retinal image blur depends both on the focus error and on the pupil diameter. Pupil size depends on luminance. To generalize our results to the real world, it is important to account for changes in pupil diameter due to luminance differences between the lab and the viewing conditions of interest.

The diameter of the blur circle in radians of visual angle is given by

θ_{b}=Aₚᵤₚᵢₜ|ΔD| (5)

where θb is the diameter of the blur circle in radians of visual angle, Aₚᵤₚᵢₗ is the pupil aperture (diameter) in meters and ΔD=D_{focus}−D_{target} is the focus error in diopters which is given by the difference between the dioptric distances of the focus and target points (see Supplement). Note that under the geometric optics approximation, the absolute value of the focus error |ΔD| in the blurry eye equals the absolute value of the interocular focus difference |ΔF| because one eye was always sharply focused (i.e. min min(|ΔD_{L}|, |ΔD_{R}|)=0.0 D) in our experiments.

The interocular delay in seconds is linearly related to each level of blur by

$$Δ t = {α}_{Δ F} \frac{{θ}_{b}}{{A}_{pupil}^{exp}} + {β}_{Δ F} \qquad ( 6 )$$

where α_{ΔF} and β_{ΔF} are the slope and constant of the best fit line to the data in FIG. 2A, and Aₚᵤₚᵢₗ^{exp} is the pupil diameter of the observer during the experiment in meters. For subsequent expressions, the constant can be dropped assuming it reflects response bias and not sensory-perceptual bias.

For a target moving at a given velocity in meters per second, a particular interocular difference in processing speed will yield an effective interocular spatial offset (i.e. position difference)

Δx=vΔt (7)

The illusory distance of the target, predicted by stereo-geometry, is given by

$$\hat{d} = \frac{I}{( I + Δ x )} d \qquad ( 8 )$$

where d is the actual distance of the target (see Supplement).

An expression for stereo-specified distance relationship can be derived by first computing the neural binocular disparity induced by the interocular delay and then converting the disparity into an estimate of depth. The binocular disparity in radians of visual angle that is induced by the position difference is given by

$$δ = \frac{Δ x}{d} \qquad ( 9 )$$

The relationship between estimated distance, binocular disparity, and actual distance is given by

$$\hat{d} = \frac{I}{( I + d δ )} d \qquad ( 10 )$$

Plugging the Eq. 9 into Eq. 10 yields Eq. 8. Thus, both ways of computing the stereo-specified distance are equivalent. Combining equations

$$\hat{d} = \frac{Id}{I + v \vert Δ F \vert {α}_{Δ F} {R}_{pupil}} \qquad ( 11 )$$

where Rₚᵤₚᵢₗ=Aₚᵤₚᵢₗ/Aₚᵤₚᵢₗ^{exp} is the ratio of the pupil diameter in the viewing conditions of interest and the pupil diameter in the lab during which the data was collected. Finally, the illusion size predicted by stereo-geometry \hat{d}-d is obtained by taking the difference between the estimated and true target distances. Eq. 11 was used to generate the predictions in FIG. 3A.

Anti-Pulfrich Monovision Corrections

Reducing the image quality of one eye with blur increases the processing speed relative to the other eye and causes the Reverse Pulfrich effect. Reducing the retinal illuminance of one eye reduces the processing speed relative to the other eye and causes the Classic Pulfrich effect. Thus, in principle, it should be possible to null the two effects by reducing the retinal illuminance of the blurry eye. Using virtual neutral density filters it was determined that the induced interocular phase disparity is given by

Δt=α_{ΔO}(ΔO)+β_{ΔO} (12)

where ΔO is the interocular difference in optical density. The optical density that should null the interocular delay of a given blurring lens is given by

$$Δ {O}_{0} = \frac{{α}_{Δ F}}{{α}_{Δ O}} Δ F \qquad ( 13 )$$

which is the interocular difference in focus error scaled by the ratio of the slopes of the best fit linear regression lines to the Reverse and Classic Pulfrich datasets. The experimental data shows (e.g., as illustrated in FIG. 4) that indeed the optical density predicted by the two regression slopes eliminates the Pulfrich effect.

Supplement: Monovision and the Misperception of Motion

Prevalence of Monovision Corrections in the United States of America

There are approximately 115 million of presbyopes in the USA. Of these, roughly 25% use contact lenses, and roughly 5% have had surgery to correct their presbyopia. Approximately 33% of the contact lens users, and approximately 50% of the surgical patients have monovision corrections. Together, this results in approximately 12.5 million adults with monovision corrections.

The disclosure may use virtual and/or real neutral density filters.

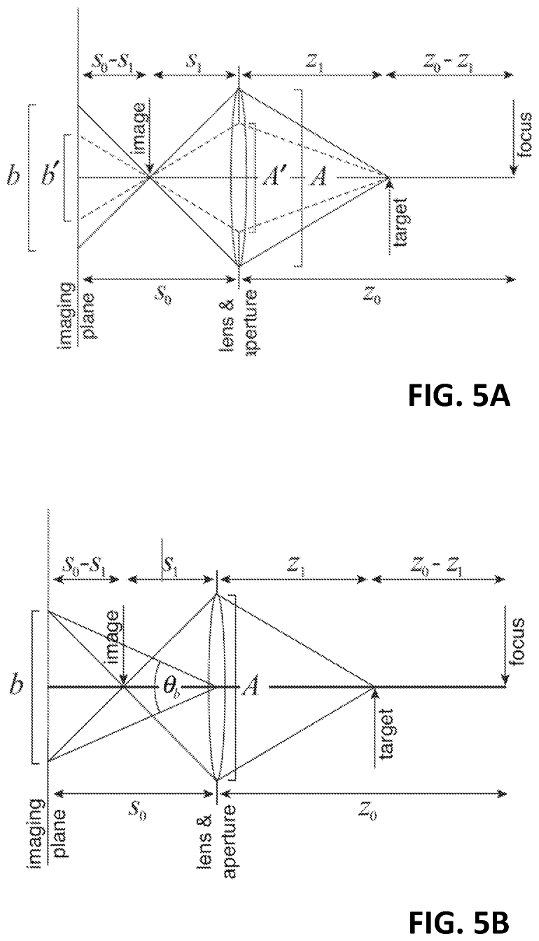

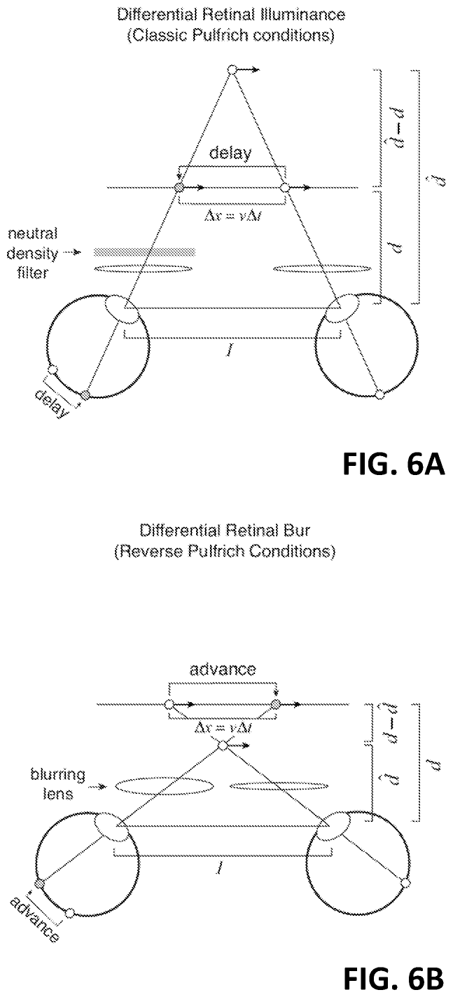

FIGS. 5A-6B show using geometric optics to relate focus error, aperture size, and blur circle size. FIG. 5A shows blur circle diameter in meters from aperture and defocus. Solid and dashed lines show how two different aperture sizes (A and A′) cause two difference blur circle sizes (b and b′) for the same focus error. FIG. 5B shows blur circle diameter in visual angle.

Relationship between focus error and defocus blur in millimeters and in visual angle Defocus (i.e. focus error) is defined as the difference in dioptric distance between the focus point and a target point (Eq. 1)

ΔD=D_{focus}−D_{target} (51)

where D_{focus} and D_{target} are the dioptric distances to the focus and target points in object space.

Diopters are defined as inverse meters, so equation 1 can be equivalently written

$$Δ D = \frac{1}{{z}_{0}} - \frac{1}{{z}_{1}} \qquad ( S2 )$$

where z₀ and z₁ are distances to the focus and target points in meters. The lens equation states that the dioptric difference in object space is equivalently given by the dioptric difference between the imaging plane and the image point in image space

$$Δ D = \frac{1}{{s}_{0}} - \frac{1}{{s}_{1}} \qquad ( S3 )$$

where s₀ and s₁ are distances to the imaging plane and image point in meters. (Note: In this derivation, we use z and s to be consistent with notational traditions in geometric optics. The symbol z₁ corresponds to the symbol d for target distance in the main text.)

Using relations between similar triangles gives the relationship

$$\frac{A}{{s}_{1}} = \frac{b}{{s}_{0} - {s}_{1}} \qquad ( S4 )$$

where A is the aperture (e.g. pupil) diameter and b is the blur circle diameter in meters (e.g., as shown in FIG. 6A).

Solving Eq. S4 for Blur Yields

$$b = A \frac{( {s}_{0} - {s}_{1} )}{{s}_{1}} = A \frac{{s}_{0} ( {s}_{0} - {s}_{1} )}{{s}_{0} {s}_{1}} \qquad ( S5 )$$

Rearranging Eq. S3 Yields

$$Δ D = \frac{( {s}_{0} - {s}_{1} )}{{s}_{0} {s}_{1}} \qquad ( S6 )$$

Multiplying Eq. S6 by negative one, taking the absolute value (e.g., because the blur circle diameter cannot be negative), and substituting into Eq. S5 gives the blur circle diameter in meters

b=As₀|ΔD| (S7)

To determine the relationship between the blur circle diameter in meters and in visual angle, it is useful to examine FIG. 5B.

From standard trigonometry, the relationship between the blur circle diameter and the subtended visual angle is

$$\frac{b / 2}{{s}_{0}} = tan ( {θ}_{b} / 2 ) \qquad ( S8 )$$

Rearranging after using the small angle approximation

b=θ_{b}s₀ (S9)

Substituting Eq. S9 into Eq. S7 and solving gives the blur circle in radians of visual angle

θ_{b}=A|ΔD| (S10)

FIGS. 6A-B show using stereo-geometry to relate interocular delay, target distance, and illusion size. FIG. 6A shows geometry predicting illusion size (\hat{d}-d) for rightward motion with a neutral density filter in front of the left eye. FIG. 6B shows stereo-geometry predicting illusion size (e.g., blur circle diameter) for rightward motion with a blurring lens in front of the left eye. It should be noted that the diagrams are not to scale.

Relationship Between Interocular Delay, Target Distance, and Illusion Size

For a given target velocity and interocular delay the effective spatial offset is given by

Δx=vΔt (S11)

where v is target velocity and Δt is interocular delay. The effective spatial offset, target velocity, and interocular delay are all signed quantities. Leftward spatial offsets, leftward velocities, and more slowly processed left-eye images are negative. Rightward spatial offsets, rightward velocities, and more quickly processed left eye images are positive.

By similar triangles, the following relationship holds

$$\frac{\hat{d}}{I} = - \frac{\hat{d} - d}{Δ x} \qquad ( S12 )$$

where \hat{d} is the estimated (i.e. illusory) target distance, d is the actual target distance, and l is the interocular distance (FIG. 6A and FIG. 6B).

Solving for the Illusory Target Distance Yields

$$\hat{d} = ( \frac{I}{I + Δ x} ) \qquad ( S13 )$$

which is Eq. 8 in the main text.

The illusion size \hat{d}-d is given by the difference between illusory and actual target distances.

### Examples

Anti-Pulfrich Monovision can be implemented with contact lenses, intraocular lenses, refractive surgery, corneal inlays, glasses, with neutral density filters or tints applied on the corrections, sunglasses, and combinations of them. The invention can be used by eye care practitioners to prescribe monovision corrections to their patients. The major vendors or distributors of ophthalmic corrections (mainly but not only contact lenses and intraocular lenses) will provide the invention to the eye care practitioners to aid them in the prescription of monovision corrections. Alternatively, the eye care practitioners will purchase the invention.

An example device may comprise a device comprising a binocular configured for anti-Pulfrich Monovision ophthalmic corrections. The device may comprise a pair of contact lenses or intraocular lenses (example not limitative). The device may comprise a first lens of the pair, fitted or implanted in eye one, with an optical power that corrects the refractive errors of that eye, therefore providing far vision in focus. The device may comprise a second lens of the pair, fitted or implanted in eye two, with an optical power that corrects the refractive errors of that eye and then adds 0.75 to 1.5 D, therefore providing near vision in focus. The second lens (in this example) may be tinted, with an optical density (e.g., between 0.05 and 0.3) such that the difference in retinal illuminance between eyes produces a Classic Pulfrich effect compensating to some extent the Reverse Pulfrich effect produced by the difference in retinal blur between eyes, and therefore reduces the misperceptions in depth of objects in motion (e.g., as illustrated in FIG. 4)

As an example, not limitative, when the patient is a driver with presbyopia, in a country were drivers use the right lane, eye one corresponds to the right eye, and eye two corresponds to the left eye.

An example method may comprise a procedure for the prescription of Anti-Pulfrich monovision for a patient. The method may be implemented by software, implemented as an App for a smartphone or a computer program in a computer. The software controls independently the two monocular images of a moving stimulus in a binocular display, generating misperceptions of blur when the subject wears a monovision correction, in the form of ophthalmic lenses or implants, or simulated with trial lenses in a trial frame or phoropter, or with adjustable lenses. The display could be 3D goggles or, as an alternative, a 3D monitor in combination with 3D glasses. The software also controls a measurement procedure of the misperception of depth in objects in motion, in this example the measurement of the Reverse Pulfrich effect, using a nulling procedure in which the patient adjust the delay until there is no perception of depth in the moving objects, while the subject looks at the display. Other procedures are possible. The Classic Pulfrich effect could be measured, alternatively or additionally to the Reverse Pulfrich effect. The software estimates the Neutral Density filter needed to generate a Classic Pulfrich effect that compensate the Reverse Pulfrich effect measured, based on the statistical knowledge gathered in other patients and in the intra-subject correlation between Reverse and Classic Pulfrich effects. The software also performs a validation procedure of the compensation. The validation procedure is very similar to the measurement procedure described herein, but in this implementation software controls a virtual neutral density filter applied to the one of the two monocular images of the binocular display. Depending on the results of the validation, the software could readjust the Neutral density filter. The validation procedure is shown is not required for the procedure to work. The combination of optical powers and optical densities of the two lenses of the pair represents the Anti-Pulfrich monovision correction for the patient.

An example system may be used for the prescription of Anti-Pulfrich monovision corrections, working in combination with a Pulfrich inducer. The system may comprise: A binocular display; and a device comprising the aforementioned procedure for the prescription of Anti-Pulfrich monovision resulting in a prescription for a patient. The Pulfrich inducer may comprise a real monovision ophthalmic correction, or simulated with trial lenses in trial frames or in a phoropter, or simulated with tunable lenses. The Pulfrich inducer may comprise filters or virtual filters implemented by the software in the binocular display. The system or kit can also check the nulling of the Pulfrich effect.

Additional Information

The following paragraphs provide further disclosure; parts of the following paragraphs may overlap in part with other disclosure elsewhere herein.

Monovision is a common prescription lens correction for presbyopia [1]. Each eye is corrected for a different distance, causing one image to be blurrier than the other. Millions of people have monovision corrections, but little is known about how interocular blur differences affect motion perception. Here, we report that blur differences cause a previously unknown motion illusion that makes people dramatically misperceive the distance and three-dimensional direction of moving objects. The effect occurs because the blurry and sharp images are processed at different speeds. For moving objects, the mismatch in processing speed causes a neural disparity, which results in the misperceptions. A variant of a 100-year-old stereo-motion phenomenon called the Pulfrich effect [2], the illusion poses an apparent paradox: blur reduces contrast, and contrast reductions are known to cause neural processing delays [3-6], but our results indicate that blurry images are processed milliseconds more quickly. We resolve the paradox with known properties of the early visual system, show that the misperceptions can be severe enough to impact public safety, and demonstrate that the misperceptions can be eliminated with novel combinations of non-invasive ophthalmic interventions. The fact that substantial perceptual errors are caused by millisecond differences in processing speed highlights the exquisite temporal calibration required for accurate perceptual estimation. The motion illusion—the reverse Pulfrich effect—and the paradigm we use to measure it should help reveal how optical and image properties impact temporal processing, an important but understudied issue in vision and visual neuroscience.

Results and Discussion

In the year 2020, nearly two billion people will have presbyopia worldwide [7]. Presbyopia is the age-related loss of focusing ability due to the stiffening of the crystalline lens inside the eye [8]. Without correction, presbyopia prevents people from reading or effectively using a smartphone.

Many corrections exist for presbyopia. Bifocals and progressive lenses are well known examples. Monovision corrections are less well known. With monovision, each eye is fitted with a lens that sharply focuses light from a different distance, providing “near vision” to one eye and “far vision” to the other. Monovision thus causes differential blur between the eyes. When users accept monovision corrections, the visual system suppresses the lower-quality image and preferentially processes the higher quality of the two images [9-11]. The consequence is an increase in effective depth of field without many of the drawbacks of other corrections (e.g., the “seam” in the visual field caused by bifocals). Unfortunately, monovision has its own drawbacks. It degrades stereoacuity [12, 13] and contrast sensitivity [14], hampering fine-scale depth discrimination and reading in low light. Monovision is also thought to cause difficulties in driving [1], and it has been implicated in an aviation accident [15]. Despite these drawbacks, many people prefer monovision corrections to other corrections, or no corrections at all [16].

Ten million people in the United States currently have monovision corrections (see STAR Methods). The number of candidates will increase in the coming years. The population is aging, and monovision is the most popular contact lens correction for presbyopia among the baby boomers [16]. A full understanding of the effects of monovision on vision is critical. However, there is no literature on how the differential blur induced by monovision impacts motion perception, which is critical for successful interaction with the environment [17].

We investigated the impact of differential blur on motion perception by measuring the Pulfrich effect, a stereo-motion phenomenon first reported nearly 100 years ago [2]. When a target oscillates horizontally in the frontoparallel plane and is viewed with unequal retinal illuminance or contrast in the two eyes [2, 18], it appears to move on an elliptical trajectory in depth (FIG. 1A). The effect occurs because the image in the eye with lower retinal illuminance or contrast is processed more slowly than the image in the other eye [2, 18, 19]. The mismatch in processing speed causes a neural binocular disparity, a difference in the effective target image locations in the two retinas [20, 21]. The disparity results in the illusory motion in depth. The Pulfrich effect has been researched extensively since its first discovery [18, 19, 22-27]. In the late 1990s and early 2000s, a flurry of work debated what the effect reveals about the neural basis of stereo and motion encoding [28-31]. But it is not known whether the Pulfrich effect is caused by the optical conditions induced by monovision corrections.

Do interocular blur differences, like interocular illuminance and contrast differences, cause misperceptions of motion? More specifically, does blur slow the speed of processing and cause a Pulfrich effect? In the classic Pulfrich effect, if the left eye retinal illuminance or contrast is decreased, observers perceive “front-left” motion (i.e., clockwise motion from above; FIG. 1A). However, we find that when the left eye is blurred, observers perceive “front-right” motion (FIG. 1B). Thus, instead of a classic Pulfrich effect, differential blur causes a reverse Pulfrich effect.

The reverse Pulfrich effect implies an apparent paradox. Blur reduces contrast and should therefore cause the blurry image to be processed more slowly, but the reverse Pulfrich effect implies that the blurry image is processed more quickly (FIG. 1C). At first, this finding appears at odds with a large body of neurophysiological and behavioral results. Low-contrast images are known to be processed more slowly in early visual cortex [4, 6, 32] and at the level of behavior [3, 5].

FIGS. 1A-D show classic and Reverse Pulfrich Effects.

FIG. 1A shows the classic Pulfrich effect. A left-eye neutral density filter causes horizontally oscillating frontoparallel motion to be misperceived in depth (i.e., “front-left”; clockwise motion from above). The image in the eye with lower retinal illuminance (gray dot) is delayed relative to the other eye (white dot), causing a neural disparity.

FIG. 1B shows the reverse Pulfrich effect. A left-eye blurring lens causes illusory motion in depth in the other direction (i.e., “front-right”). The blurrier image (gray dot) is advanced relative to the other eye (white dot), causing a neural disparity with the opposite sign.

FIG. 1C shows neural image positions across time for the classic Pulfrich effect, no Pulfrich effect, and the reverse Pulfrich effect.

The paradox is resolved by recognizing two facts. First, blur reduces the contrast of high-spatial-frequency image components more than low-frequency image components [33-35]. Second, extensive neurophysiological [6, 32, 36, 37] and behavioral [3, 5] literatures indicate that high spatial frequencies are processed more slowly than low spatial frequencies, all else equal. Together, these facts suggest that the blurry image is processed more quickly than the sharp image because the high spatial frequencies in the sharp image decrease the speed at which it is processed. Thus, the reverse Pulfrich effect can be explained by known properties of the early visual system.

Psychophysical Results

To measure the reverse Pulfrich effect, we performed a one-interval two-alternative forced choice (2AFC) experiment. We used trial lenses to induce interocular differences in blur and a haploscope for dichoptic presentation of moving targets (FIG. 2A). On each trial, a target oscillated from left to right (or right to left) while the observer fixated a central dot. The onscreen interocular delay of the target images was under experimenter control. If the onscreen delay is zero, onscreen disparity specifies that the target is moving in the plane of the screen. If the onscreen delay is non-zero, onscreen disparity specifies that the target is moving on an elliptical trajectory outside the plane of the screen. Observers reported whether the target was moving leftward or rightward when it appeared to be in front of the screen. Human observers made these judgments easily and reliably.

For a given difference in focus error, we measured the proportion of trials that observers reported “front right” as a function of the onscreen interocular delay. In each condition, performance was summarized with the point of subjective equality (PSE), the 50% point on the psychometric function (FIG. 2B and FIG. 2C). The PSE specifies the onscreen delay required to make the target appear to move in the plane of the screen (i.e., no motion in depth).

FIGS. 2D-F show reverse, Classic, and Anti-Pulfrich Conditions: Psychophysical Data

FIG. 2D shows binocular stimulus. The target was a horizontally moving 0.25°×1.0° white bar. Arrows show motion, speed, and direction, and dashed bars show bar positions during a trial; both are for illustrative purposes only and were not in the actual stimulus. Observers reported whether they saw three-dimensional (3D) target motion as front-right or front-left with respect to the screen. Fuse the two half-images to perceive the stimulus in 3D. Cross and divergent fusers will perceive the bar nearer and farther than the screen, respectively.

FIG. 2E shows points of subjective equality (PSEs) for one observer, expressed as onscreen interocular delay relative to baseline. Interocular differences in focus error (bottom axis, white circles) cause the reverse Pulfrich effect. Interocular differences in retinal illuminance (top axis, gray squares) cause the classic Pulfrich effect. Appropriately tinting the blurring lens (light gray circles) can eliminate the motion illusions and act as an anti-Pulfrich correction. (In the anti-Pulfrich conditions, optical density was different for each observer and focus difference.) Shaded regions indicate bootstrapped standard errors. Best-fit regression lines are also shown.

FIG. 2F shows psychometric functions for seven of the reverse Pulfrich conditions in FIG. 2E. Arrows indicate raw PSEs.

FIG. 2G shows psychometric functions for seven of the thirteen tested differential blur conditions (top) and all five of the tested differential retinal illuminance conditions (bottom). The arrows indicate the PSE for each condition.

FIG. 2H shows points of subjective equality (PSEs), expressed as interocular delay, as a function of interocular differences in focus error (bottom axis, white circles) or as a function of differences in retinal illuminance for one human observer (top axis, gray squares). Differences in focus error were introduced by defocusing each eye from 0.0 D to 1.5 D, while keeping the other eye sharply focused. Differences in retinal illuminance were induced by placing neutral density filters in front of one eye, while leaving the other eye unfiltered (black squares). The best fit regression lines are also shown.

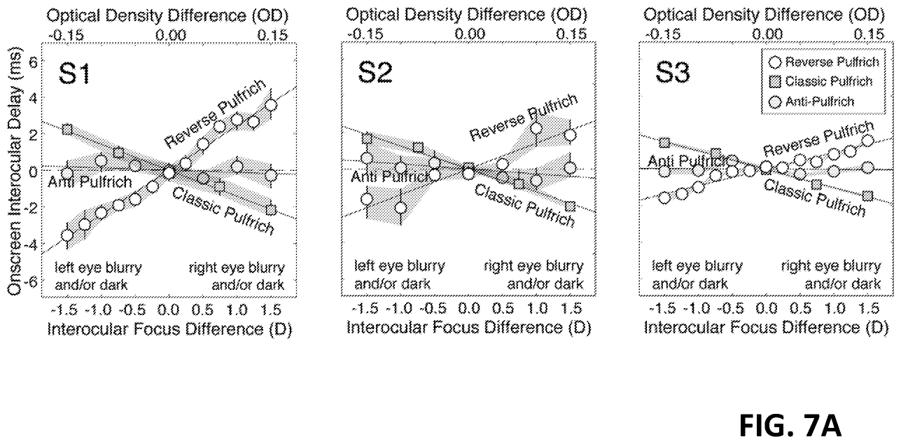

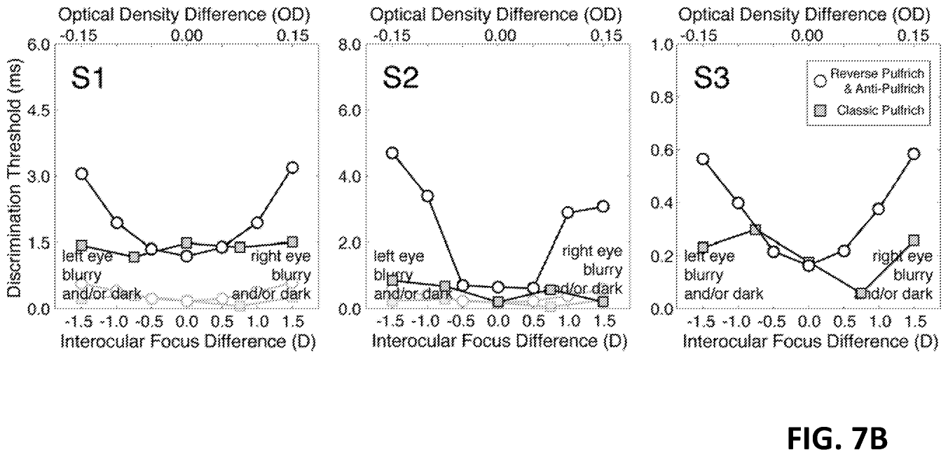

See also FIGS. 7A-B.

The magnitude of the reverse Pulfrich effect increases with the difference in focus error between the eyes (FIG. 2E, white circles; FIGS. 7A-B). (Discrimination thresholds also increase with differences in focus error [12]; FIG. 2F and FIGS. 7A-B). Negative differences indicate conditions in which the left-eye retinal image is blurry and the right-eye retinal image is sharp. In these conditions, the left-eye onscreen image must be delayed (i.e., negative PSE shift) for the target to be perceived as moving in the screen plane. Positive differences in focus error indicate that the left-eye retinal image is sharp and the right-eye retinal image is blurry. In these conditions, the right-eye onscreen image must be delayed (i.e., positive PSE shift). The results indicate that the blurrier image is processed faster. For the first observer, a ±1.5 D difference in focus error caused an interocular difference in processing speed of ±3.7 ms (FIG. 2E).

As a control, we measured the classic Pulfrich effect. Systematically reducing the retinal illuminance to one eye, while leaving the other eye unperturbed, reverses the pattern of PSE shifts (FIG. 2E, gray squares; FIGS. 7A-B). When the left eye's retinal illuminance is reduced, the left-eye onscreen image must be advanced in time for the target to be perceived as moving in the plane of the screen, and vice versa. Consistent with classic findings, these results indicate that the darker image is processed more slowly than the brighter image.

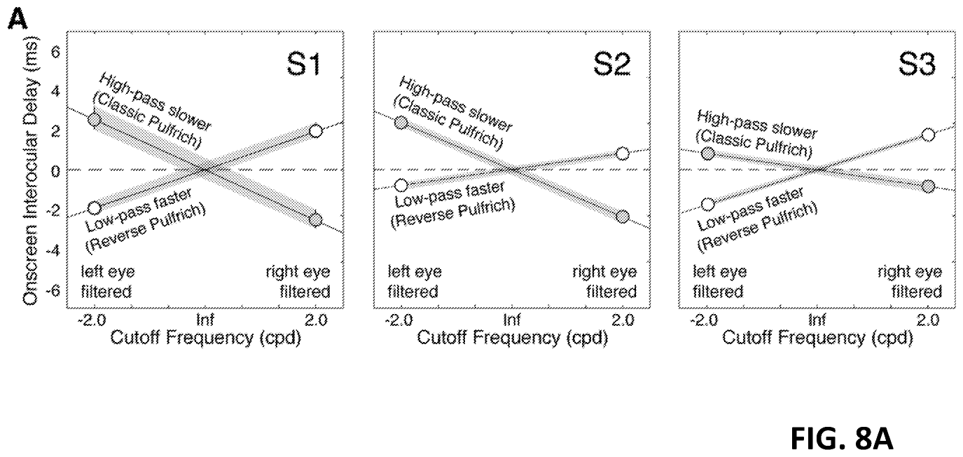

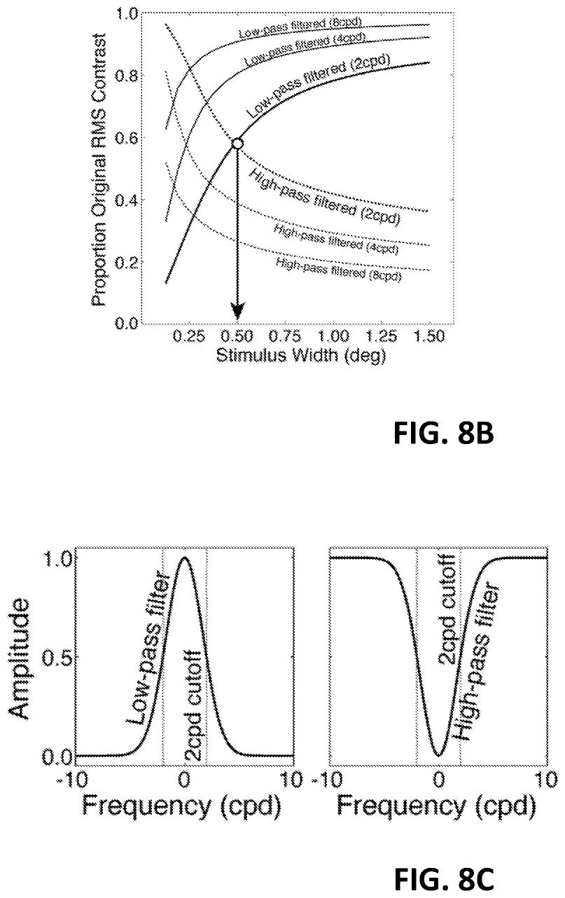

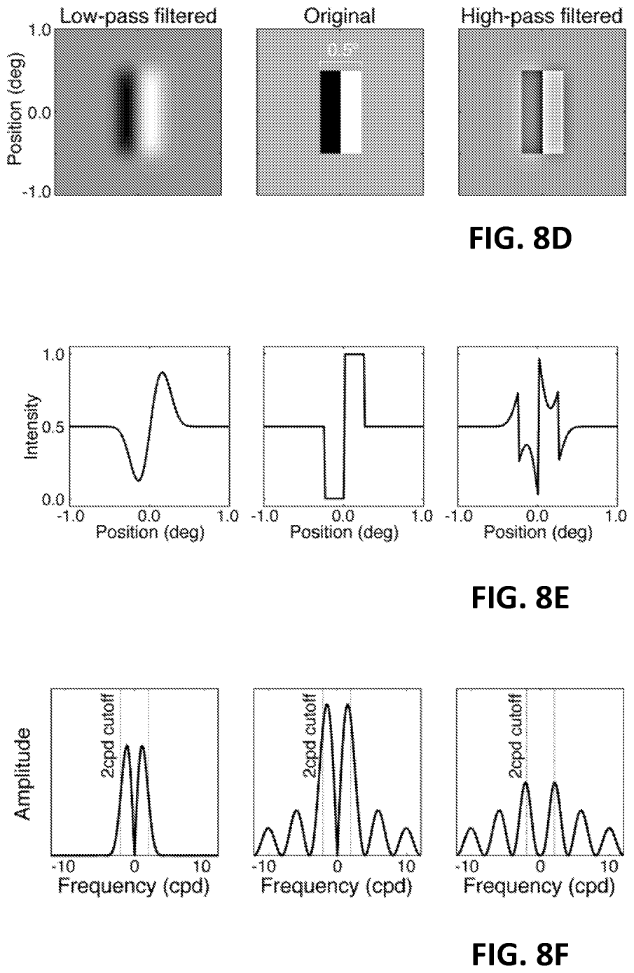

Why does the reverse Pulfrich effect occur? To test the hypothesis that high spatial frequencies in the sharp image slow down its processing (see above), we ran an additional experiment. In the first condition, the onscreen stimulus to one eye was high-pass filtered while the other stimulus was unperturbed. High-pass filtering sharpens the image by removing low frequencies, increases the average spatial frequency, and should decrease the processing speed relative to the original unperturbed stimulus. In the second condition, the onscreen stimulus to one eye was low-pass filtered (FIG. 3D and FIG. 3E). Low-pass filtering removes high frequencies, approximates the effects of optical blur, and should increase processing speed. Results with high- and low-pass filtered stimuli should therefore resemble the classic and reverse Pulfrich effects, respectively. This prediction is confirmed by the data (FIG. 3F and FIGS. 8A-F). Importantly, the interocular differences in processing speed cannot be attributed to luminance or contrast differences because the filtered stimuli were designed to have identical luminance and contrast (FIGS. 8A-F). The computational rules that relate frequency content to processing speed remain to be worked out and should make a fruitful area for future study. A full understanding of these rules may facilitate the construction of stimuli that yield larger effect sizes than those reported here [37].

The performance of the first human observer is consistent across all observers and experiments (FIG. 3G, FIGS. 7A-B, and FIGS. 8A-F). The interocular differences in processing speed were 1.4-3.7 ms across observers for 1.5 D differences in focus error and 1.5-2.1 ms for 0.150 D differences in retinal illuminance. Similar effects are obtained with low- and high-pass filtering. These differences in processing speed may appear modest. However, a difference of a few milliseconds in processing speed can lead to dramatic illusions in depth (see below).

Effect sizes vary across observers but appear correlated in each observer across conditions (FIG. 3G). A larger pool of observers is necessary to confirm this trend. Future studies should measure the range and determine the origin of these interobserver differences. Developing techniques that increase the speed of data collection will aid these efforts [38].

FIGS. 3D-G shows spatial Frequency Filtering: Psychophysical Data

FIG. 3D shows original stimuli were composed of adjacent black-white (top) or white-black (bottom) 0.25°×1.00° bars.

FIG. 3E shows high-pass or low-pass filtered stimuli (shown only for black-white bar stimuli). High- and low-pass filtered stimuli were designed to have identical luminance and contrast (see FIGS. 8A-F).

FIG. 3F shows resulting interocular delays. High-pass filtered stimuli are processed more slowly, and low-pass filtered stimuli are processed more quickly than the original unfiltered stimulus. Negative cutoff frequencies indicate that the left eye was filtered (high or low pass). Positive cutoff frequencies indicate that the right eye was filtered.

FIG. 3G shows effect sizes for each human observer in multiple conditions, obtained from the best-fit regression lines (see FIG. 2E and FIG. 3F). FIG. 3G shows maximum interocular differences in processing speed for three different human observers in monovision-like optical conditions (white bars; 1.5 D interocular focus difference) vs. darkening one eye with a neutral density filter (gray bars; +0.15 optical density). Differences in processing speed for anti-Pulfrich conditions are also shown. Two manipulations resulted in reverse Pulfrich effects (white bars): blurring one eye (left) and low-pass filtering one eye (right). Two manipulations resulted in classic Pulfrich effects (gray bars): darkening one eye (left) and high-pass filtering one eye (right). A fifth manipulation—appropriately darkening the blurring lens (left, small light-gray bars)—eliminates the Pulfrich effect and acts as an anti-Pulfrich correction.

Motion Illusions in the Real World

Monovision corrections cause misperceptions of motion. How large will these misperceptions be in daily life? If the illusions are small, they can be safely ignored. If the illusions are large, they may have serious consequences. To predict the severity of misperceptions in real-world viewing, differences in pupil size, target speed, and viewing distance must be taken into account (see STAR Methods). The same focus error causes less blur with smaller pupils. The same interocular difference in processing speed causes larger neural disparities at faster speeds. The same disparity specifies larger depths at longer viewing distances.

Consider a target object, five meters away, moving from left to right in daylight conditions. Predicted illusion sizes with different monovision corrections strengths are shown for one observer as a function of target speed (FIG. 3A). A +1.5 D difference in optical power (far lens over left eye), a common monovision correction strength [1], will cause the distance of a target moving at 15 miles per hour to be overestimated by 2.8 m. This, remarkably, is the width of a narrow street lane! If the prescription is reversed (−1.5 D; far lens over right eye) target distance will be underestimated by 1.3 m. Also, illusion sizes should increase with faster target speeds, stronger monovision corrections, and dimmer lighting conditions [19, 23, 24, 39] (e.g., driving at dawn, dusk, or night; see STAR Methods).

Illusions this large will not only be disturbing for the person wearing the monovision correction; they may compromise public safety. In countries where motorists drive on the right side of the road (e.g., the US), cars and cyclists in the near lane of cross traffic move from left to right. Placing the far lens in the left eye will cause distance overestimation, which may result in casual braking and increase the likelihood of traffic accidents (FIG. 3B). Placing the far lens in the right eye may be advisable. Doing so should result in distance underestimation and more cautious braking, which may reduce the likelihood of collisions (FIG. 3C). In countries where motorists drive on the left side of the road (e.g., the United Kingdom), the opposite practice should be considered (i.e., far lens in left eye). The current standard is to place the far lens in the dominant eye [1, 41], but this practice does not improve patient acceptance rate, patient satisfaction [41, 42], or quantitative measures of visual performance [13, 43]. The scenarios described here may invite reexamination of standard ophthalmic practice.

In the real world, many cues exist that tend to indicate the correct rather than illusory depths. The literature on cue combination [44, 45] suggests that in cue rich situations, the magnitude of the reverse Pulfrich effect may be somewhat reduced from the predictions in FIG. 4A. Determining which cues are most important [46] and examining how the reverse Pulfrich effect manifests in real-world viewing conditions will be of clinical and scientific interest. These issues could be examined with virtual- or augmented-reality headsets that provide precise programmatic control of near-photorealistic graphical renderings.

FIG. 3A-C Show Monovision Corrections and Real-World Misperceptions of Depth

FIG. 3A shows illusion size as a function of speed for an object moving from left to right at 5.0 m, with different monovision corrections strengths (curves). Monovision correction strengths (interocular focus difference, ΔF) typically rFange between 1.0 D and 2.0 D [1]. Shaded regions show speeds associated with jogging, cycling, and driving. Illusion sizes are predicted from stereo-geometry, assuming a pupil size (2.1 mm) that is typical for daylight conditions [39] and interocular delays that were measured from observer 51 (see FIG. 2E). The predictions assume that the observer can focus the target at 5.0 m in one eye [40].

FIG. 3B shows the distance of cross traffic moving from left to right will be overestimated when the left eye is focused far (sharp) and the right eye is focused near (blurry).

FIG. 3C shows the distance of left-to-right cross traffic will be underestimated when the left and right eyes are focused near and far, respectively.

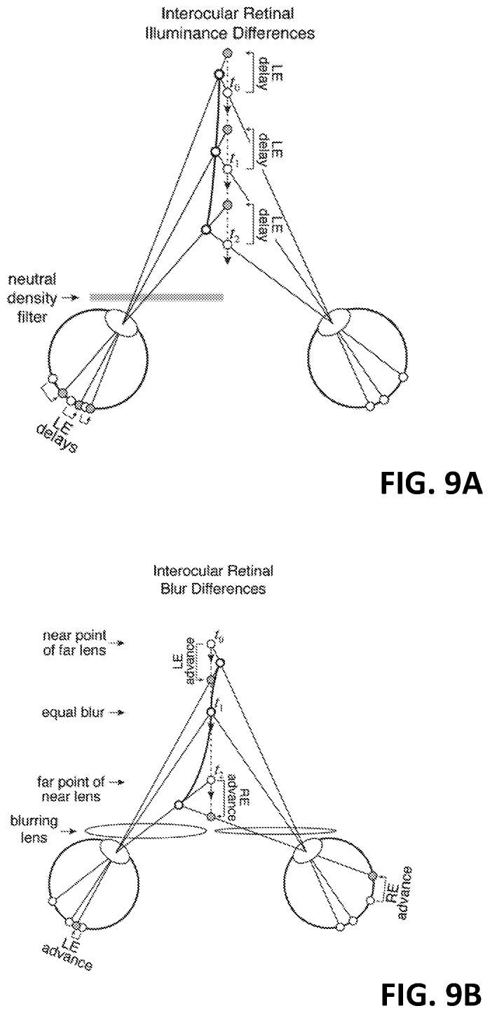

See also FIGS. 9A-B.

Another implication of these results is that objects moving toward an observer along straight lines should appear to follow S-curve trajectories (FIGS. 9A-B). These misperceptions should make it difficult to play tennis, baseball, and other ball sports requiring accurate perception of moving targets. Monovision corrections should be avoided when playing these sports.

Eliminating Monovision-Induced Motion Illusions

Reconsidering prescribing practices is one approach to minimizing the consequences of monovision-induced motion illusions, but it is not the perfect solution. It would be far preferable to eliminate the illusions altogether. Because increased blur and reduced retinal illuminance have opposite effects on processing speed, it should be possible to null the two effects by tinting the blurring lens. We reran the original experiment with appropriately tinted blurring lenses for each human observer (see STAR Methods). This “anti-Pulfrich correction” eliminates the motion illusion in all human observers (FIG. 2E and FIG. 3G). Of course, for a given monovision prescription, the lens forming the blurry image varies with target distance. Anti-Pulfrich monovision corrections thus cannot work at all target distances. Tinting the near lens (blurry, dark images for far targets; sharp, dark images for near targets) will eliminate the Pulfrich effect for far targets but exacerbate it for near targets. However, because many presbyopes retain some accommodation and use it to focus the distance-corrected eye [40], the range of far distances for which motion misperceptions may be eliminated can be quite large: 0.67 m to the horizon for a presbyope with 1.5 D of residual accommodation. Given that accurate perception of moving targets is probably more important for tasks at far than at near distances (e.g., driving versus reading), tinting the near lens is likely to be the preferred solution. This issue, however, clearly needs further study.

Adaptation

Previous studies have shown that blur perception changes with consistent exposure to blur [47]. Do motion illusions change over time as patients adapt to monovision corrections? The literature on adaptation to the classic Pulfrich effect may provide a guide [22, 24, 48, 49]. However, in these adaptation studies, the eye with the dark image was fixed. With monovision corrections, the eye with the blurry image varies with target distance. Thus, it is unclear whether observers will adapt away motion illusions caused by differential blur. This is an important area for future study, both for basic science and for the development of successful clinical interventions.

Spatial-Frequency Binding Problem

Scientific discoveries often present new scientific opportunities. We have argued that the reverse Pulfrich effect occurs because sharp images contain more high frequencies (i.e., fine details) than blurry images and because high frequencies are processed more slowly than low frequencies. Indeed, different spatial frequencies are processed in early visual cortex with different latencies [37]. Thus, the frequency components of an image should appear to split apart when a target object moves, causing rigidly moving images to appear non-rigid. This percept is not typically experienced. To achieve a unified percept, the visual system must therefore have a mechanism for binding the different frequency components together.

Variants of the paradigm that we have used to measure the reverse Pulfrich effect have great potential for investigating the visual system's solution to the spatial-frequency binding problem. The measurements have exquisite temporal precision, often to within fractions of a millisecond (FIG. 2E, FIG. 2F, FIG. 3F, and FIG. 3G). This precision should prove useful for studying this fundamentally important but understudied problem in vision and visual neuroscience.

We have reported a new version of a 100-year-old illusion: the reverse Pulfrich effect. We found that interocular differences in image blur, like those caused by monovision corrections, cause millisecond interocular differences in processing speed. For moving targets, these differences cause dramatic illusions of motion in depth. The fact that mismatches of a few milliseconds can cause substantial misperceptions highlights how exquisitely the visual system must be calibrated for accurate percepts to occur. The fact that these misperceptions are rare indicates how well the visual system is calibrated under normal circumstances.

Star*Methods

### REFERENCES

1. Evans, B. J. W. (2007). Monovision: a review. Ophthalmic Physiol. Opt. 27, 417-439.
2. Pulfrich, C. (1922). Die Stereoskopie im Dienste der isochromen and heterochromen Photometrie. Naturwissenschaften 10, 553-564.
3. Nachmias, J. (1967). Effect of exposure duration on visual contrast sensitivity with square-wave gratings. J. Opt. Soc. Am. 57, 421-427.
4. Shapley, R. M., and Victor, J. D. (1978). The effect of contrast on the transfer properties of cat retinal ganglion cells. J. Physiol. 285, 275-298.
5. Levi, D. M., Harwerth, R. S., and Manny, R. E. (1979). Suprathreshold spatial frequency detection and binocular interaction in strabismic and anisometropic amblyopia. Invest. Ophthalmol. Vis. Sci. 18, 714-725.
6. Albrecht, D. G. (1995). Visual cortex neurons in monkey and cat: effect of contrast on the spatial and temporal phase transfer functions. Vis. Neurosci. 12, 1191-1210.
7. Fricke, T. R., Tahhan, N., Resnikoff, S., Papas, E., Burnett, A., Ho, S. M., Naduvilath, T., and Naidoo, K. S. (2018). Global prevalence of presbyopia and vision impairment from uncorrected presbyopia: systematic review, meta-analysis, and modelling. Ophthalmology 125, 1492-1499.
8. Charman, W. N. (2008). The eye in focus: accommodation and presbyopia. Clin. Exp. Optom. 91, 207-225.
9. Westendorf, D. H., Blake, R., Sloane, M., and Chambers, D. (1982). Binocular summation occurs during interocular suppression. J. Exp. Psychol. Hum. Percept. Perform. 8, 81-90.
10. Schor, C., Landsman, L., and Erickson, P. (1987). Ocular dominance and the interocular suppression of blur in monovision. Am. J. Optom. Physiol. Opt. 64, 723-730.
11. Zheleznyak, L., Sabesan, R., Oh, J.-S., MacRae, S., and Yoon, G. (2013). Modified monovision with spherical aberration to improve presbyopic through-focus visual performance. Invest. Ophthalmol. Vis. Sci. 54, 3157-3165.
12. Westheimer, G., and McKee, S. P. (1980). Stereoscopic acuity with defocused and spatially filtered retinal images. J. Opt. Soc. Am. A 70, 772-778.
13. McGill, E., and Erickson, P. (1988). Stereopsis in presbyopes wearing monovision and simultaneous vision bifocal contact lenses. Am. J. Optom. Physiol. Opt. 65, 619-626.
14. Pardhan, S., and Gilchrist, J. (1990). The effect of monocular defocus on binocular contrast sensitivity. Ophthalmic Physiol. Opt. 10, 33-36.
15. Nakagawara, V. B., and Véronneau, S. J. (2000). Monovision contact lens use in the aviation environment: a report of a contact lens-related aircraft accident. Optometry 71, 390-395.
16. Bennett, E. S. (2008). Contact lens correction of presbyopia. Clin. Exp. Optom. 91, 265-278.
17. Burge, J., and Geisler, W. S. (2015). Optimal speed estimation in natural image movies predicts human performance. Nat. Commun. 6, 7900.
18. Reynaud, A., and Hess, R. F. (2017). Interocular contrast difference drives illusory 3D percept. Sci. Rep. 7, 5587.
19. Lit, A. (1949). The magnitude of the Pulfrich stereophenomenon as a function of binocular differences of intensity at various levels of illumination. Am. J. Psychol. 62, 159-181.
20. Wheatstone, C. (1838). On some remarkable, and hitherto unobserved, phenomena of binocular vision. Philos. Trans. R. Soc. Lond. 128, 371-394.
21. Burge, J., and Geisler, W. S. (2014). Optimal disparity estimation in natural stereo images. J. Vis. 14, 1.
22. Standing, L. G., Dodwell, P. C., and Lang, D. (1968). Dark adaptation and the pulfrich effect. Percept. Psychophys. 4, 118-120.
23. Wilson, J. A., and Anstis, S. M. (1969). Visual delay as a function of luminance. Am. J. Psychol. 82, 350-358.
24. Rogers, B. J., and Anstis, S. M. (1972). Intensity versus adaptation and the Pulfrich stereophenomenon. Vision Res. 12, 909-928.
25. Morgan, M. J., and Thompson, P. (1975). Apparent motion and the Pulfrich effect. Perception 4, 3-18.
26. Carney, T., Paradiso, M. A., and Freeman, R. D. (1989). A physiological correlate of the Pulfrich effect in cortical neurons of the cat. Vision Res. 29, 155-165.
27. Lages, M., Mamassian, P., and Graf, E. W. (2003). Spatial and temporal tuning of motion in depth. Vision Res. 43, 2861-2873.
28. Qian, N., and Andersen, R. A. (1997). A physiological model for motion-stereo integration and a unified explanation of Pulfrich-like phenomena. Vision Res. 37, 1683-1698.
29. Read, J. C. A., and Cumming, B. G. (2005). All Pulfrich-like illusions can be explained without joint encoding of motion and disparity. J. Vis. 5, 901-927.
30. Read, J. C. A., and Cumming, B. G. (2005). The stroboscopic Pulfrich effect is not evidence for the joint encoding of motion and depth. J. Vis. 5, 417-434.
31. Qian N, Freeman R D. Pulfrich phenomena are coded effectively by a joint motion-disparity process. Journal of Vision 9, 24.
32. Bair, W., and Movshon, J. A. (2004). Adaptive temporal integration of motion in direction-selective neurons in macaque visual cortex. J. Neurosci. 24, 7305-7323.
33. Campbell, F. W., and Green, D. G. (1965). Optical and retinal factors affecting visual resolution. J. Physiol. 181, 576-593.
34. Navarro, R., Artal, P., and Williams, D. R. (1993). Modulation transfer of the human eye as a function of retinal eccentricity. J. Opt. Soc. Am. A 10, 201-212.
35. Burge, J., and Geisler, W. S. (2011). Optimal defocus estimation in individual natural images. Proc. Natl. Acad. Sci. USA 108, 16849-16854.
36. Breitmeyer, B. G., and Ganz, L. (1977). Temporal studies with flashed gratings: inferences about human transient and sustained channels. Vision Res. 17, 861-865.
37. Vassilev, A., Mihaylova, M., and Bonnet, C. (2002). On the delay in processing high spatial frequency visual information: reaction time and VEP latency study of the effect of local intensity of stimulation. Vision Res. 42, 851-864.
38. Bonnen, K., Burge, J., Yates, J., Pillow, J., and Cormack, L. K. (2015). Continuous psychophysics: target-tracking to measure visual sensitivity. J. Vis. 15, 14.
39. Stockman, A., and Sharpe, L. T. (2006). Into the twilight zone: the complexities of mesopic vision and luminous efficiency. Ophthalmic Physiol. Opt. 26, 225-239.
40. Almutairi, M. S., Altoaimi, B. H., and Bradley, A. (2018). Accommodation in early presbyopes fit with bilateral or unilateral near add. Optom. Vis. Sci. 95, 43-52.
41. Wolffsohn, J. S., and Davies, L. N. (2019). Presbyopia: effectiveness of correction strategies. Prog. Retin. Eye Res. 68, 124-143.
42. Schor, C., Carson, M., Peterson, G., Suzuki, J., and Erickson, P. (1989). Effects of interocular blur suppression ability on monovision task performance. J. Am. Optom. Assoc. 60, 188-192.
43. Erickson, P., and Schor, C. (1990). Visual function with presbyopic contact lens correction. Optom. Vis. Sci. 67, 22-28.
44. Landy, M. S., Maloney, L. T., Johnston, E. B., and Young, M. (1995). Measurement and modeling of depth cue combination: in defense of weak fusion. Vision Res. 35, 389-412.
45. Ernst, M. O., and Banks, M. S. (2002). Humans integrate visual and haptic information in a statistically optimal fashion. Nature 415, 429-433.
46. Burge, J., and Jaini, P. (2017). Accuracy maximization analysis for sensory-perceptual tasks: computational improvements, filter robustness, and coding advantages for scaled additive noise. PLoS Comput. Biol. 13, e1005281.
47. Radhakrishnan, A., Dorronsoro, C., Sawides, L., Webster, M. A., and Marcos, S. (2015). A cyclopean neural mechanism compensating for optical differences between the eyes. Curr. Biol. 25, R188-R189.
48. Wolpert, D. M., Miall, R. C., Cumming, B., and Boniface, S. J. (1993). Retinal adaptation of visual processing time delays. Vision Res. 33, 1421-1430.
49. Plainis, S., Petratou, D., Giannakopoulou, T., Radhakrishnan, H., Pallikaris, LG., and Charman, W. N. (2013). Interocular differences in visual latency induced by reduced-aperture monovision. Ophthalmic Physiol. Opt. 33, 123-129.
50. Brainard, D. H. (1997). The psychophysics toolbox. Spat. Vis. 10, 433-436.
51. United States Census Bureau. U.S. and world population clock, https://www.census.gov/popclock.
52. Cope, J. R., Collier, S. A., Rao, M. M., Chalmers, R., Mitchell, G. L., Richdale, K., Wagner, H., Kinoshita, B. T., Lam, D. Y., Sorbara, L., et al. (2015). Contact lens wearer demographics and risk behaviors for contact lens-related eye infections—United States, 2014. MMWR Morb. Mortal. Wkly. Rep. 64, 865-870.
53. Morgan, P. B., Woods, C. A., Tranoudis, I. O., Efron, N., Jones, L., Aighamdi, W., Nair, V., Merchan, N. L., Teufl, I./M., Grupcheva, C. N., et al. (2019). International contact lens prescribing in 2018. Contact Lens Spectr. 34, 26-32.
54. National Eye Institute. Cataracts defined tables, https://nei.nih.gov/eyedata/cataract/tables.
55. Ingenito, K. (2015). Premium cataract options gain ground. Ophthalmol. Manage. 19, 42-43.

Star*Methods

Experimental Model and Subject Details

Three human observers ran in the experiment; two were authors. All human observers had normal or corrected to normal visual acuity (20/20), a history of isometropia, and normal stereoacuity as confirmed by the Titmus Stereo Test. The observers were aged 24, 29, 40 years old and had refractive errors of −4.75 D, 0.00 D, and 0.00 D diopters, respectively, at the time of the measurements. Two observers were males; the other was female. The experimental protocols were approved by the Institutional Review Board at the University of Pennsylvania and were in compliance with the Declaration of Helsinki.

Method Details

Prevalence of Monovision Corrections

There are approximately 123 million presbyopes in the USA [51]. Approximately 12.9 million of these presbyopes wear contact lenses, and 4.5 million (35%) of these contact lens wearers have monovision corrections [52, 53]. Approximately 30 million presbyopes have had surgery to implant intraocular lenses [54], and approximately 5.1 million (17%) of these surgical patients have received monovision corrections [55]. Together, this results in approximately 9.6 million presbyopes with monovision corrections in the USA.

Apparatus

Stimuli were displayed on a custom-built four-mirror haploscope. Left- and right-eye images were presented on two identical VPixx VIEWPixx LED monitors. Monitors were calibrated (i.e., the gamma functions were linearized) using custom software routines. The monitors had a size of 52.2×29.1 cm, spatial resolution of 1920×1080 pixels, a native refresh rate of 120 Hz, and a maximum luminance of 105.9 cd/m². The maximum luminance after light loss due to mirror reflections was 93.9 cd/m². The monitors were daisy-chained together and controlled by the same AMD FirePro D500 graphics card with 3 GB GDDR5 VRAM to ensure that the left and right eye images were presented synchronously. Custom firmware was written so that each monitor was driven by a single color channel; the red channel drove the left monitor and the green channel drove the right monitor. The single-channel drive to each monitor was then split to all three channels to enable gray scale presentation. Simultaneous measurements with two optical fibers connected to an oscilloscope confirmed that the left and right eye monitor refreshes occurred within ˜5 microseconds of one another.

Human observers viewed the monitors through mirror cubes with 2.5 cm circular openings positioned one inter-ocular distance apart. Heads were stabilized with a chin and forehead rest. The haploscope mirrors were adjusted such that the vergence distance matched the distance of the monitors. The light path from monitor to eye was 100 cm, as confirmed both by a laser ruler measurement and by a visual comparison with a real target at 100 cm. At this distance, each pixel subtended 1.09 arcmin. Stimulus presentation was controlled via the Psychophysics Toolbox-3 [50]. Anti-aliasing enabled sub-pixel resolution permitting accurate presentations of disparities as small as 15-20 arcsec.

Stimuli

The target stimulus was a binocularly presented, horizontally moving, white vertical bar (FIG. 2D). The target bar subtended 0.25°×1.00° of visual angle. In each eye, the image of the bar moved left and right with a sinusoidal profile. An interocular phase shift between the left- and right-eye images introduced a spatial disparity between the left- and right-eye bars. The left- and right-eye onscreen bar positions were given by

x_{L}(t)=E cos(2πωt+ϕ₀+ϕ) (Equation ST1a)

x_{R}(t)=E cos(2πωt+ϕ₀) (Equation ST1b)

where x_{L} and x_{R} are the left and right eye x-positions in degrees of visual angle, E is the movement amplitude in degrees of visual angle, co is the temporal frequency, ϕ₀ is the starting phase which in our experiment determines whether the target starts on the left or the right side of the display, t is time, and ϕ is the phase shift between the images.

The interocular temporal shift (i.e., delay or advance) in seconds associated with a particular phase shift is

Δt=ϕ/(2πω) (Equation ST2)

Negative values indicate the left eye onscreen image is delayed relative to the right; positive values indicate the left eye onscreen image is advanced relative to the right.

When the interocular temporal shift equals zero, the virtual bar moves in the frontoparallel plane at the distance of the monitors. When the temporal shift is non-zero, a spatial binocular disparity results, and the virtual bar follows a near-elliptical trajectory of motion in depth. The binocular disparity in radians of visual angle as a function of time is given by

$$δ ( t ) = {x}_{R} ( t ) - {x}_{L} ( t ) = 2 E sin ( \frac{ϕ}{2} ) sin ( 2 π ω t + {ϕ}_{0} = \frac{ϕ}{2} ) \qquad ( Equation ST3 )$$

Here, negative disparities are crossed and positive disparities are uncrossed, indicating that the target is nearer and farther than the screen distance, respectively. The disparity takes on its maximum magnitude when the perceived stimulus is directly in front of the observer and the lateral movement is at its maximum speed. When the stimulus is moving to the right, the maximum disparity in visual angle is given by δₘₐₓ=2E sin(ϕ/2).

In our experiment, the movement amplitude was 2.5° of visual angle (i.e., 5.0° total change in visual angle in each direction), the temporal frequency was 1 cycle/s, and the starting phase ϕ₀ was randomly chosen to be either 0 or π. Restricting the starting phase to these two values forced the stimuli to start either 2.5° to the right or 2.5° to the left of center on each trial. The onscreen interocular phase shift ranged between ±216 arcmin at maximum, corresponding to interocular delays of ±10.0 ms. The range and particular values were adjusted to the sensitivity of each human observer.

Two sets of five vertical 0.25°×1.00° bars in a “picket fence” arrangement flanked the region of the screen traversed by the target bar. The picket fences were defined by disparity to be at the screen distance, and served as a stereoscopic reference for the observer. A 1/f noise texture, also defined by disparity to be at the screen distance, covered the periphery of the display to aid binocular fusion. A small fixation dot marked the center of the screen.

Procedure

The observer's task was to report whether the target bar was moving leftward or rightward when it appeared to be nearer than the screen on its virtual trajectory in depth. Observers fixated the fixation dot throughout each trial. Using a one-interval two-alternative forced choice procedure, nine-level psychometric functions were collected in each condition using the method of constant stimuli. Each function was fit with a cumulative Gaussian using maximum likelihood methods. The 50% point on the psychometric function—the point of subjective equality (PSE)—indicates the onscreen interocular delay needed to null the interocular difference in processing speed. The pattern of PSEs across conditions was fit via linear regression, yielding a slope and y-intercept. Average y-intercepts were nearly zero for each observer: 0.06 ms, −0.06 ms, and 0.01 ms, respectively. To emphasize the differences in slope (i.e., the changes in processing speed in the slope) induced by interocular perturbations, we zeroed the y-intercepts when plotting the PSE data. Observers responded to 180 trials per condition in counter-balanced blocks of 90 trials each.

Defocus and Blur

The interocular focus difference is the magnitude of the defocus in the right eye minus the magnitude of the defocus in the left eye

ΔF=|ΔD_{R}|−|ΔD_{L}| (Equation ST4)

where ΔD=D_{focus}−D_{target} is the defocus, the difference between the dioptric distances of the focus and target points. To manipulate the amount of defocus blur in each eye, we positioned trial lenses ˜12 mm from each eye, centered on each optical axis, between each eye and the front of the mirror cubes of the haploscope.

Human observers ran in thirteen conditions defined by interocular focus difference. (One observer, S2, ran in only seven). Each eye was myopically defocused from 0.00 D to 1.50 D in 0.25 D steps while the other eye was kept sharp. The first six conditions defocused the left eye (0.25 D to 1.50 D in 0.25 D steps) while leaving the right eye sharp (ΔF<0.0 D). In the seventh condition, both eyes were sharp (ΔF=0.0 D). The final six conditions defocused the right eye (0.25 D to 1.50 D in 0.25 D steps) while leaving the left eye sharp (ΔF<0.0 D).

In the condition in which both eyes were sharply focused, the optical distances of the left- and right-eye monitors were set to optical infinity with +1.00 D trial lenses. All human observers indicated that they could sharply focus the monitor when they fully relaxed the accommodative power of their eyes. Because each trial lens absorbs a small fraction of the incident light, having a trial lens in front of each eye in all conditions ensures that retinal illuminance is matched in both eyes in all conditions. To induce interocular differences in focus error, we placed a stronger positive lens (i.e., +1.25 D, +1.50 D, +1.75 D, +2.00 D) in front of one eye. This procedure puts one eye's monitor beyond optical infinity, thus introducing myopic focus errors that cannot be cleared by accommodation. Before each run, the observer viewed a test target to confirm that he/she could clearly focus a target at optical infinity in the 0.0 D baseline condition. Undercorrected hyperopia or overcorrected myopia could place the far point of each eye beyond optical infinity, frustrating our attempts to control the optical conditions. To protect against this possibility, before running each observer, we estimated the far points of the eyes with standard optometric techniques. Then, if necessary, we adjusted the trial lens power so that the monitors were positioned at the desired optical distance.

Another potential concern is that the eyes could accommodate independently to clear the blur in each eye. However, there are several reasons to think that differential blur was successfully induced. First, positioning the optical distance of one monitor beyond optical infinity (see above) minimizes the possibility that differential optical power could be compensated by differential accommodation. Second, accommodation in the two eyes tends to be strongly coupled, especially for targets straight ahead [8, 40]. Third, discrimination thresholds (d′=1.0) increase systematically with interocular difference in focus error, which is consistent with the literature showing that differential blur deteriorates stereoacuity [12] (FIG. 2F and FIGS. 7A-B).

Neutral Density Filters

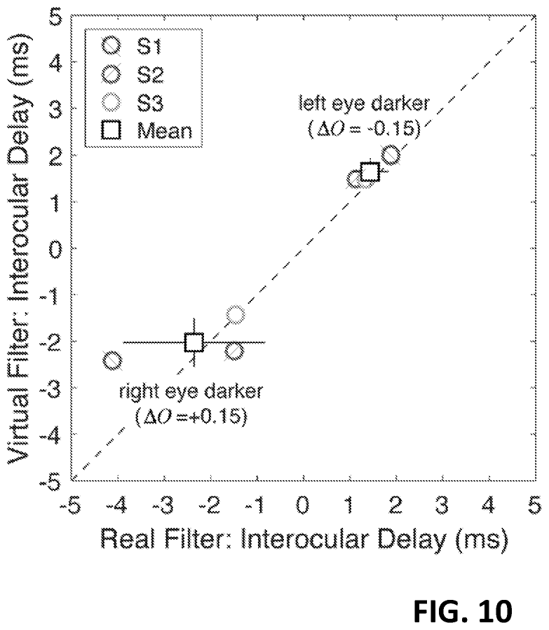

To induce interocular differences in retinal illuminance we placed ‘virtual’ neutral density filters in front of the eyes. To do so, we converted optical density to transmittance, the proportion of incident light that is passed through the filter, using the standard expression T=10^{-OD} where T is transmittance and OD is optical density. Then, we reduced the luminance of one eye's monitor by a scale factor equal to the transmittance. In all observers, performance with real and equivalent virtual neutral density filters is essentially identical, suggesting that the virtual filters were implemented accurately (FIG. 10).

The interocular difference in optical density ΔO=OD_{R}−OD_{L} is the difference between the optical density of filters placed over the right and left eyes. Human observers ran in five conditions with virtual neutral density filters, with equally spaced interocular differences in optical density between −0.15 and 0.15. Two conditions introduced a filter in front of the left eye (ΔO<0.00). In one condition, both eyes were unfiltered (ΔO=0.00). And two other conditions introduced a filter in front of the right eye (ΔO>0.00).

Low- and High-Pass Spatial Filtering

To test the hypothesis that the reverse Pulfrich effect is caused by differences in the processing speed of different spatial frequencies, we filtered the onscreen stimulus of one eye with two different frequency filters. The low-pass filter was Gaussian-shaped

k_{low}=exp[−0.5(f/σ_{f})²] (Equation ST5)

with a standard deviation of σ_{f}=f₀\sqrt{ln 4} set by the cutoff frequency f₀ so that the filter reached half-height at f₀ (i.e., 2 cpd in the current experiments; see FIG. 3). The high-pass filter complemented the low-pass filter and was given by

k_{high}=1−k_{low} (Equation ST6)

After high-pass filtering, the mean luminance was added back in so that the high-pass and low-pass filtered stimuli had the same mean luminance.

To isolate the impact of spatial frequency content on processing speed, we modified the onscreen stimulus from the main experiment. Rather than the 0.25°×1.00° white bar, the onscreen stimulus was changed to a 0.50°×1.00° stimulus that was composed of adjacent 0.25°×1.0° black and white (or white and black) bars (FIG. 3D). This modification ensured that the low- and high-pass filtered stimuli had identical luminance and identical contrast (see FIGS. 8A-F). Each human observer collected 180 trials in each of eight conditions—low-versus high-pass filtering, left- versus right-eye filtered, black-white versus white-black stimulus types—collected in counter-balanced order. Black-white versus white-black stimulus types had little impact so results were collapsed across stimulus type.

Generalizing Results to the Real World

To predict the motion misperceptions that monovision will cause in the real world, it is important to account for the differences in viewing conditions that may impact illusion sizes. Although the experimental conditions were chosen based on differences in focus error, the reverse Pulfrich effect is more directly mediated by differences in image blur. The amount of retinal image blur in each eye depends both on the focus error and on the pupil diameter. Thus, it is important to account for changes in pupil diameter that will be caused by luminance differences between the lab and the viewing conditions of interest.

The blur circle diameter in radians of visual angle is given by

θ_{b}=A|ΔD| (Equation ST7)

where θ_{b} is the diameter of the blur circle in radians of visual angle and A is the pupil aperture (diameter) in meters. In our experiments, we assumed a pupil diameter of 2.5 mm, corresponding to the luminance in the experiment [39]. Under the geometrical optics approximation, the absolute value of the defocus |ΔD| in the blurry eye equals the absolute value of the interocular focus difference WI because one eye was always sharply focused (i.e., min |ΔD_{L}|, |ΔD_{R}|=0.0 D) in our experiments.

The interocular delay in seconds is linearly related to each level of blur by

$$Δ t = {α}_{Δ F} \frac{{θ}_{b}}{{A}_{exp}} + {β}_{Δ F} \qquad ( Equation ST8 )$$

where α_{ΔF} and β_{ΔF} are the slope and y-intercept of the best-fit line to the data in FIG. 2E, and Aₑₓₚ is the pupil diameter of the observer in meters during the experiment. The constant (i.e., y-intercept) can be dropped assuming it reflects response bias and not sensory-perceptual bias.

For a target moving at a given velocity in meters per second, a particular interocular difference in processing speed will yield an effective interocular spatial offset (i.e., position difference)

Δx=vΔt (Equation ST9)

The illusory distance of the target, predicted by stereo-geometry, is given by

$$\hat{d} = \frac{I}{( I + Δ x )} d \qquad ( Equation ST10 )$$

where I is the inter-pupillary distance and d is the actual distance of the target. Combining Equations ST7-ST10 yields a single expression for the illusory distance

$$\hat{d} = ( \frac{I}{I + v \vert Δ F \vert R {α}_{Δ F}} ) d \qquad ( Equation ST11 )$$

where R=A/Aₑₓₚ is the ratio between the pupil diameters in the viewing condition of interest and in the lab when the psychophysical data was collected. Finally, taking the difference between the illusory and actual target distances \hat{d}-d yields the illusion size (see FIG. 3A).

The expression for the illusory distance can also be derived by first computing the neural binocular disparity caused by the delay-induced position difference, and then converting the disparity into an estimate of depth. The binocular disparity in radians of visual angle is given by

$$δ = \frac{Δ x}{d} \qquad ( Equation ST12 )$$

The relationship between illusory distance, binocular disparity, and actual distance is given by

$$\hat{d} = \frac{I}{( I + d δ )} d \qquad ( Equation ST13 )$$

Plugging Equation ST12 into Equation ST13 yields Equation ST10. Thus, both methods of computing the illusory distance are equivalent.

Anti-Pulfrich Monovision Corrections

Reducing the image quality of one eye with blur increases the processing speed relative to the other eye and causes the reverse Pulfrich effect. Reducing the retinal illuminance of one eye reduces the processing speed relative to the other eye and causes the classic Pulfrich effect. Thus, in principle, it should be possible to null the two effects by reducing the retinal illuminance of the blurry eye. The interocular delay in seconds is linearly related to each interocular difference in optical density Δ by

Δt=α_{ΔO}(ΔO)+β_{ΔO} (Equation ST14)

The optical density that should null the interocular delay of a given interocular focus difference is given by

$$Δ \text{?} = - \frac{{α}_{Δ F}}{\text{?}} Δ F \\ \text{?} \text{indicates text missing or illegible when filed} \qquad ( Equation ST15 )$$

the interocular difference in focus error scaled by the ratio of the slopes of the best-fit regression lines to the reverse and classic Pulfrich datasets. The optical density predicted by the two regression slopes eliminates the Pulfrich effect (FIG. 2E and FIG. 3A).

Quantification and Statistical Analysis

All experiments were performed in MATLAB 2017b using Psychtoolbox (version 3.0.12) [50]. All analyses were performed in MATLAB 2017b. Psychophysical data are presented for each individual human observer. Cumulative Gaussian fits of the psychometric functions were in good agreement with the raw data. Bootstrapped standard errors are reported on all data points unless otherwise noted.

FIG. 7A-B show reverse, classic, and anti-Pulfrich conditions: Interocular delays and discrimination thresholds. FIGS. 7A-B may provide additional information for FIGS. 2A-F. FIG. 7A shows reverse, classic, and anti-Pulfrich effects. Interocular differences in focus error cause the reverse Pulfrich effect; the blurrier image is processed more quickly. Interocular differences in retinal illuminance cause the classic Pulfrich effect; the darker image is processed more slowly. In the anti-Pulfrich condition, the blurry image is darkened to eliminate interocular delay (see Methods). FIG. 7B shows Discrimination thresholds. Thresholds for each observer (d′=1.0) in the reverse Pulfrich conditions (interocular focus differences) and the anti-Pulfrich conditions (interocular focus differences plus retinal illuminance differences) were similar and were thus averaged together (white circles). In each human observer, discrimination thresholds increased systematically with differences in interocular blur, consistent with the classic literature on how blur differences deteriorate stereoacuity[S1]. These threshold functions thus provide evidence that the desired optical conditions were achieved. To reduce clutter, bootstrapped 95% confidence intervals are not plotted. In all cases but one, the confidence interval is smaller than the data point. Discrimination threshold in the classic Pulfrich conditions (i.e. interocular retinal illuminance differences only) are also shown (gray squares). Differences in retinal illuminance up to +0.150 D had no systematic effect on thresholds. (Note: the y-axis has a different scale for each observer to emphasize the similarities in the threshold patterns. To give a sense of scale, the classic Pulfrich data from observer S3, the most sensitive observer, is re-plotted in the subplots for observers S1 and S2; faint circles and squares.)

FIGS. 8A-F show spatial frequency filtered stimuli: Interocular delays and stimulus construction. FIGS. 8A-F may provide additional information for FIGS. 3D-G. FIG. 8A Interocular delays with high- and low-pass filtered stimuli for each human observer. The onscreen image for one eye was filtered and the image for the other eye was left unperturbed. High-pass filtered images were processed slower than the unperturbed images, similar to how reduced retinal illuminances induces the classic Pulfrich effect. Low-pass filtered images were processed faster than unperturbed images, similar to how optical blur induces the reverse Pulfrich effect. FIG. 8B Proportion of original stimulus contrast after low-pass filtering vs. high-pass filtering (solid vs. dashed curves, respectively) as a function of total black-white (or white-black) bar width. The white circle and arrow indicate the stimulus width (0.5°) that equates the root-mean-squared (RMS) contrast of the stimulus after low- and high-pass filtering. Because low-pass and high-pass filtered images had identical luminance and contrast, the differential effects in FIG. 8A cannot be attributed to luminance or contrast. FIG. 8C shows low-pass and high-pass filters with a 2 cpd cutoff frequency. FIG. 8D low-pass filtered stimulus, original stimulus, and high-pass filtered stimulus with matched luminance and contrast. FIG. 8E shows horizontal intensity profiles of the stimuli in FIG. 8D. FIG. 8F shows amplitude spectra of the horizontal intensity profiles in FIG. 8E. Note how, for each stimulus type, the peak of the lowest frequency lobe shifts relative to the cutoff frequency of the filters.

FIGS. 9A-B show misperception of motion towards the observer. FIGS. 9A-B may provide additional information for FIGS. 3A-C. FIG. 9A shows predicted perceived motion trajectory (bold curve), given target motion directly towards the observer (dashed line), with an interocular retinal illuminance difference. Here, a neutral density filter in front of the left eye causes its image to be processed more slowly, regardless of target distance. Stereo-geometry predicts that the target will appear to travel along a curved trajectory that bends towards the darkened eye (bold curve) rather than in a straight line[S2]. FIG. 9B shows predicted perceived motion trajectory, given target motion directly towards the observer, with an interocular blur difference. The left eye is corrected for near and the right eye is corrected for far. The eye that is processed more quickly now changes systematically as a function of target distance. When the target is far, the left eye image will be blurry and be processed more quickly. When the target arrives at an intermediate distance where both eyes will form equally blurry images, the processing will be the same in both eyes and the target will appear to move directly towards the observer. When the target is near, the right eye image will be blurry and processed more quickly. The resulting illusory motion will trace an S-curve trajectory as the target traverses the distances between the near point of the far lens and the far point of the near lens. Even more striking effects occur for targets moving towards and to the side of the observer, along oblique motion trajectories. A full description of these effects, however, is beyond the scope of the current paper. (Note: the diagrams are not to scale.)

FIG. 10 shows real and virtual neutral density filters: Interocular delays. Related to STAR Methods-Neutral Density Filters. Real and virtual neutral density filters with the same optical densities (i.e. 0.150 D; 71% transmittance) caused similar delays for all human observers (colored circles) and the mean human observer (black square). Interocular differences in optical density, AO, are negative when the left eye retinal illuminance is reduced and positive when the right eye retinal illuminance is reduced. Error bars indicate standard deviations. The results suggest that the software implementation of the virtual neutral density filters was accurate.

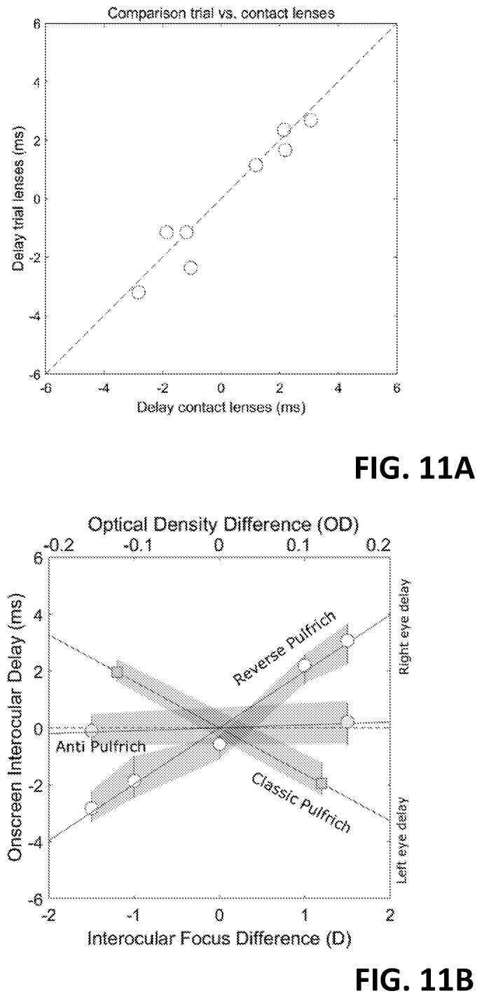

FIGS. 11A-B show data indicating that the reverse Pulfrich effect and the anti-Pulfrich effect manifests with contact lenses. FIG. 11A shows a comparison of delay trial lenses and delay contact lenses. FIG. 11B shows optical density difference, interocular focus difference, and onscreen interocular delay for contact lenses.

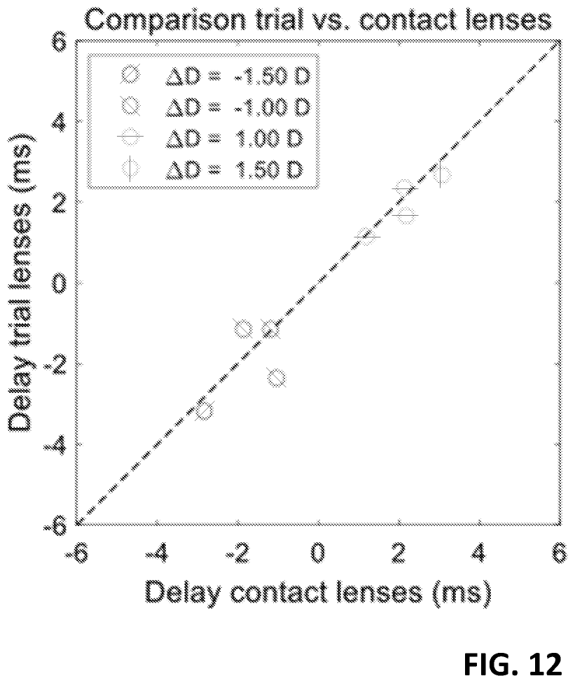

FIG. 12 shows the similarity of measured effects with blur differences induced by contact lenses (clinically relevant) and trial lenses (used in the original experiment).

### SUPPLEMENTAL REFERENCES

S1. Westheimer G, McKee S P. Stereoscopic acuity with defocused and spatially filtered retinal images. J Opt Soc Am A 1980; 70:772-8.

S2. Spiegler J B. Apparent path of a pulfrich target as a function of the slope of its plane of notion. Am J Optom Physiol Opt 1986; 63:209-16.

The present disclosure may comprise at least the following aspects.

Aspect 1. An ophthalmic device (e.g., or system), comprising, consisting of, or consisting essentially of: a first lens having a first optical characteristic that modifies a distance of a focal point of a first eye; and a second lens having a second optical characteristic that modifies a distance of a focal point of a second eye, wherein the distance of the focal point of the first eye modified by the first lens is different than the distance of the focal point of the second eye modified by the second lens, and wherein the second lens has a third optical characteristic to reduce a misperception of a distance of a moving object.

Aspect 2. The ophthalmic device of Aspect 1, wherein the first lens does not have the third optical characteristic or has less of the third optical characteristic than the second lens.

Aspect 3. The ophthalmic device of any one of Aspects 1-2, wherein one or more of the first lens or the second lens comprises one or more of a contact lens, an intraocular lens, an ocular implant, an ocular inlay, an ocular onlay, a lens mounted on a wearable frame, or a virtual lens formed by the addition, removal, or reshaping of ocular media.

Aspect 4. The ophthalmic device of any one of Aspects 1-3, wherein the first lens corrects refractive errors of the first eye and the second lens corrects refractive errors of the second eye.

Aspect 5. The ophthalmic device of any one of Aspects 1-4, wherein the second lens corrects refractive errors of the second eye and has additional refractive power of one or more of about 0.75 to 1.5 diopters, about 0.5 to about 1.5 diopters, or about 0.5 to about 2.0 diopters.

Aspect 6. The ophthalmic device of any one of Aspects 1-5, wherein the third optical characteristic comprises one or more of a tinting, a filter, a density filter, or a neutral density filter.

Aspect 7. The ophthalmic device of any one of Aspects 1-6, wherein an optical density of the third optical characteristic of the second lens is between about 0.05 and about 0.3.

Aspect 8. A method comprising, consisting of, or consisting essentially of: outputting a first representation of a moving object to a first eye of a user; outputting a second representation of the moving object to a second eye of the user, wherein the second representation is viewed by the second eye via a lens that modifies a focal point of the second eye to be different than a focal point of the first eye; receiving data indicative of an adjustment to a characteristic of one or more of the first representation or the second representation; determining, based on the data indicative of the adjustment, a lens characteristic associated with reducing a misperception of distance of the moving object; and outputting data indicative of the lens characteristic.

Aspect 9. The method of Aspect 8, wherein receiving data indicative of the adjustment comprises receiving data indicative of an adjustment that prevents the user from having a perception of depth in the moving object.

Aspect 10. The method of any one of Aspects 8-9, wherein determining the lens characteristic comprises determining one or more of an optical density, a tinting, a density filter, a virtual filter, a virtual density filter, or a neutral density filter.

Aspect 11. The method of any one of Aspects 8-10, wherein the lens characteristic is associated with eliminating the misperception of distance of the moving object.

Aspect 12. The method of any one of Aspects 8-11, wherein the first representation comprises a first monocular image of a binocular display and the second representation comprises a second monocular image of the binocular display.

Aspect 13. The method of any one of Aspects 8-12, further comprising updating, based on the data indicative of the adjustment, one or more of the first representation or the second representation to reduce the misperception of distance of the moving object.

Aspect 14. The method of any one of Aspects 8-13, wherein the first eye views the first representation via the first lens of any one of Aspects 1-7 (e.g., or Aspects 21-28), and wherein the second eye views the second representation via the second lens of any one of Aspects 1-7.

Aspect 15. The method of any one of Aspects 8-14, wherein the first representation is viewed by the first eye via an additional lens.

Aspect 16. The method of Aspect 15, wherein the additional lens modifies a distance of focal point of the first eye such that the distance of the focal point of the first eye as modified by the additional lens is different from the distance of the focal point of the second eye as modified by the lens.

Aspect 17. The method of any one of Aspects 15-16, further comprising providing one or more of the lens or the additional lens to the user.

Aspect 18. The method of any one of Aspects 8-17, wherein the lens characteristic comprises one or more of an optical characteristic of the lens that modifies the focal point of the second eye or an optical characteristic of an additional lens for the first eye.

Aspect 19. A device comprising, consisting of, or consisting essentially of: one or more processors; and a memory storing instructions that, when executed by the one or more processors, cause the device to perform the method of any one of Aspects 8-18.

Aspect 20. A non-transitory computer-readable medium storing instructions that, when executed by one or more processors, cause a device to perform the method of any one of Aspects 8-19.

Aspect 21. An ophthalmic device comprising, consisting of, or consisting essentially of a first lens having a first optical characteristic that modifies a distance of a focal point of a first eye of a wearer of the first lens, wherein the distance of the focal point of the first eye as modified by the first lens is different than a distance of a focal point of a second eye of the wearer, wherein the first lens has a second optical characteristic to reduce a misperception of a distance of a moving object.

Aspect 22. The ophthalmic device of Aspect 21, further comprising a second lens that modifies a distance of a focal point of a second eye, wherein the second lens one or more of: does not have the second optical characteristic or has a different amount of the second optical characteristic than the second lens.

Aspect 23. The ophthalmic device of any one of Aspects 21-22, wherein the first lens comprises one or more of a contact lens, an intraocular lens, an ocular implant, an ocular inlay, an ocular onlay, a lens mounted on a wearable frame, or a virtual lens formed by the addition, removal, or reshaping of ocular media.

Aspect 24. The ophthalmic device of any one of Aspects 21-23, wherein the addition, removal, or reshaping of ocular media is due to corneal laser refractive surgery.

Aspect 25. The ophthalmic device of any one of Aspects 21-24, wherein the first lens corrects refractive errors of the first eye.

Aspect 26. The ophthalmic device of any one of Aspects 21-25, wherein the first lens corrects refractive errors of the first eye and has additional refractive power of one or more of about 0.75 to 1.5 diopters, about 0.5 to about 1.5 diopters, or about 0.5 to about 2.0 diopters.

Aspect 27. The ophthalmic device of any one of Aspects 21-26, wherein the second optical characteristic comprises one or more of a tinting, a filter, a density filter, or a neutral density filter.

Aspect 28. The ophthalmic device of any one of Aspects 21-27, wherein an optical density of the second optical characteristic of the first lens is between about 0.05 and about 0.3.

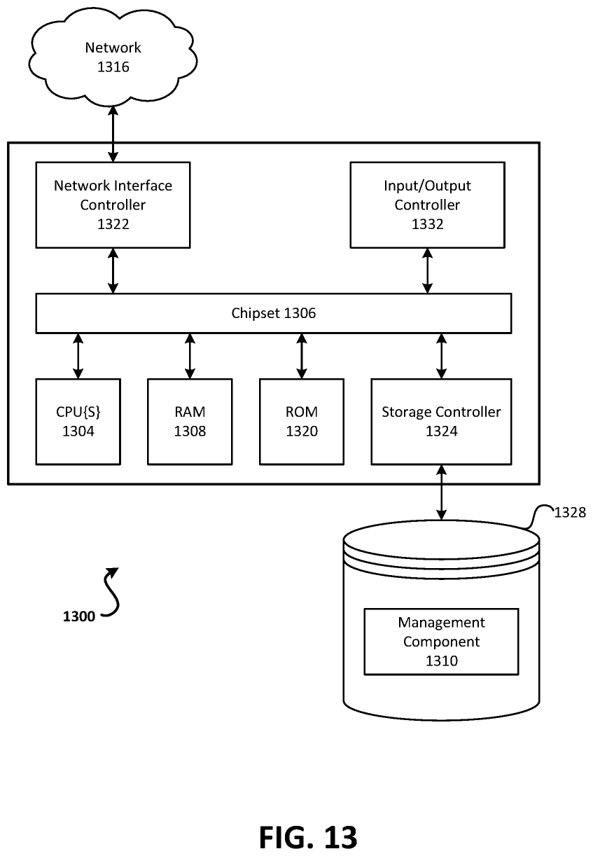

FIG. 13 depicts a computing device that may be used in various aspects, such as the ophthalmic devices described. The computer architecture shown in FIG. 13 shows a conventional server computer, workstation, desktop computer, laptop, tablet, network appliance, PDA, e-reader, digital cellular phone, or other computing node, and may be utilized to execute any aspects of the computers described herein, such as to implement the methods described herein.

The computing device 1300 may include a baseboard, or “motherboard,” which is a printed circuit board to which a multitude of components or devices may be connected by way of a system bus or other electrical communication paths. One or more central processing units (CPUs) 1304 may operate in conjunction with a chipset 1306. The CPU(s) 1304 may be standard programmable processors that perform arithmetic and logical operations necessary for the operation of the computing device 1300.

The CPU(s) 1304 may perform the necessary operations by transitioning from one discrete physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits including registers, adders-subtractors, arithmetic logic units, floating-point units, and the like.

The CPU(s) 1304 may be augmented with or replaced by other processing units, such as GPU(s) 1305. The GPU(s) 1305 may comprise processing units specialized for but not necessarily limited to highly parallel computations, such as graphics and other visualization-related processing.

A chipset 1306 may provide an interface between the CPU(s) 1304 and the remainder of the components and devices on the baseboard. The chipset 1306 may provide an interface to a random access memory (RAM) 1308 used as the main memory in the computing device 1300. The chipset 1306 may further provide an interface to a computer-readable storage medium, such as a read-only memory (ROM) 1320 or non-volatile RAM (NVRAM) (not shown), for storing basic routines that may help to start up the computing device 1300 and to transfer information between the various components and devices. ROM 1320 or NVRAM may also store other software components necessary for the operation of the computing device 1300 in accordance with the aspects described herein.

The computing device 1300 may operate in a networked environment using logical connections to remote computing nodes and computer systems through local area network (LAN) 1316. The chipset 1306 may include functionality for providing network connectivity through a network interface controller (NIC) 1322, such as a gigabit Ethernet adapter. A NIC 1322 may be capable of connecting the computing device 1300 to other computing nodes over a network 1316. It should be appreciated that multiple NICs 1322 may be present in the computing device 1300, connecting the computing device to other types of networks and remote computer systems.

The computing device 1300 may be connected to a mass storage device 1328 that provides non-volatile storage for the computer. The mass storage device 1328 may store system programs, application programs, other program modules, and data, which have been described in greater detail herein. The mass storage device 1328 may be connected to the computing device 1300 through a storage controller 1324 connected to the chipset 1306. The mass storage device 1328 may consist of one or more physical storage units. A storage controller 1324 may interface with the physical storage units through a serial attached SCSI (SAS) interface, a serial advanced technology attachment (SATA) interface, a fiber channel (FC) interface, or other type of interface for physically connecting and transferring data between computers and physical storage units.

The computing device 1300 may store data on a mass storage device 1328 by transforming the physical state of the physical storage units to reflect the information being stored. The specific transformation of a physical state may depend on various factors and on different implementations of this description. Examples of such factors may include, but are not limited to, the technology used to implement the physical storage units and whether the mass storage device 1328 is characterized as primary or secondary storage and the like.

For example, the computing device 1300 may store information to the mass storage device 1328 by issuing instructions through a storage controller 1324 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computing device 1300 may further read information from the mass storage device 1328 by detecting the physical states or characteristics of one or more particular locations within the physical storage units.

In addition to the mass storage device 1328 described above, the computing device 1300 may have access to other computer-readable storage media to store and retrieve information, such as program modules, data structures, or other data. It should be appreciated by those skilled in the art that computer-readable storage media may be any available media that provides for the storage of non-transitory data and that may be accessed by the computing device 1300.

By way of example and not limitation, computer-readable storage media may include volatile and non-volatile, transitory computer-readable storage media and non-transitory computer-readable storage media, and removable and non-removable media implemented in any method or technology. Computer-readable storage media includes, but is not limited to, RAM, ROM, erasable programmable ROM (“EPROM”), electrically erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage, other magnetic storage devices, or any other medium that may be used to store the desired information in a non-transitory fashion.

A mass storage device, such as the mass storage device 1328 depicted in FIG. 13, may store an operating system utilized to control the operation of the computing device 1300. The operating system may comprise a version of the LINUX operating system. The operating system may comprise a version of the WINDOWS SERVER operating system from the MICROSOFT Corporation. According to further aspects, the operating system may comprise a version of the UNIX operating system. Various mobile phone operating systems, such as IOS and ANDROID, may also be utilized. It should be appreciated that other operating systems may also be utilized. The mass storage device 1328 may store other system or application programs and data utilized by the computing device 1300.

The mass storage device 1328 or other computer-readable storage media may also be encoded with computer-executable instructions, which, when loaded into the computing device 1300, transforms the computing device from a general-purpose computing system into a special-purpose computer capable of implementing the aspects described herein. These computer-executable instructions transform the computing device 1300 by specifying how the CPU(s) 1304 transition between states, as described above. The computing device 1300 may have access to computer-readable storage media storing computer-executable instructions, which, when executed by the computing device 1300, may perform the methods described herein.

A computing device, such as the computing device 1300 depicted in FIG. 13, may also include an input/output controller 1332 for receiving and processing input from a number of input devices, such as a keyboard, a mouse, a touchpad, a touch screen, an electronic stylus, or other type of input device. Similarly, an input/output controller 1332 may provide output to a display, such as a computer monitor, a flat-panel display, a digital projector, a printer, a plotter, or other type of output device. It will be appreciated that the computing device 1300 may not include all of the components shown in FIG. 13, may include other components that are not explicitly shown in FIG. 13, or may utilize an architecture completely different than that shown in FIG. 13.

As described herein, a computing device may be a physical computing device, such as the computing device 1300 of FIG. 13. A computing node may also include a virtual machine host process and one or more virtual machine instances. Computer-executable instructions may be executed by the physical hardware of a computing device indirectly through interpretation and/or execution of instructions stored and executed in the context of a virtual machine.

It is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Components are described that may be used to perform the described methods and systems. When combinations, subsets, interactions, groups, etc., of these components are described, it is understood that while specific references to each of the various individual and collective combinations and permutations of these may not be explicitly described, each is specifically contemplated and described herein, for all methods and systems. This applies to all aspects of this application including, but not limited to, operations in described methods. Thus, if there are a variety of additional operations that may be performed it is understood that each of these additional operations may be performed with any specific embodiment or combination of embodiments of the described methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded on a general-purpose computer, special-purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto may be performed in other sequences that are appropriate. For example, described blocks or states may be performed in an order other than that specifically described, or multiple blocks or states may be combined in a single block or state. The example blocks or states may be performed in serial, in parallel, or in some other manner. Blocks or states may be added to or removed from the described example embodiments. The example systems and components described herein may be configured differently than described. For example, elements may be added to, removed from, or rearranged compared to the described example embodiments.

It will also be appreciated that various items are illustrated as being stored in memory or on storage while being used, and that these items or portions thereof may be transferred between memory and other storage devices for purposes of memory management and data integrity. Alternatively, in other embodiments, some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

While the methods and systems have been described in connection with preferred embodiments and specific examples, it is not intended that the scope be limited to the particular embodiments set forth, as the embodiments herein are intended in all respects to be illustrative rather than restrictive.

It will be apparent to those skilled in the art that various modifications and variations may be made without departing from the scope or spirit of the present disclosure. Other embodiments will be apparent to those skilled in the art from consideration of the specification and practices described herein. It is intended that the specification and example figures be considered as exemplary only, with a true scope and spirit being indicated by the following claims.

1. An ophthalmic device comprising:
a first lens having a first optical characteristic that modifies a distance of a focal point of a first eye of a wearer of the first lens, wherein the distance of the focal point of the first eye as modified by the first lens is different than a distance of a focal point of a second eye of the wearer, wherein the first lens has a second optical characteristic to reduce a misperception of a distance of a moving object.

2. The ophthalmic device of claim 1, further comprising a second lens that modifies a distance of a focal point of a second eye, wherein the second lens one or more of: does not have the second optical characteristic or has a different amount of the second optical characteristic than the second lens.

3. The ophthalmic device of claim 1, wherein the first lens comprises one or more of a contact lens, an intraocular lens, an ocular implant, an ocular inlay, an ocular onlay, a lens mounted on a wearable frame, or a virtual lens formed by addition, removal, or reshaping of ocular media.

4. (canceled)

5. The ophthalmic device of claim 1, wherein the first lens corrects refractive errors of the first eye.

6. The ophthalmic device of claim 1, wherein the first lens corrects refractive errors of the first eye and has additional refractive power of one or more of about 0.75 to 1.5 diopters, about 0.5 to about 1.5 diopters, or about 0.5 to about 2.0 diopters.

7. The ophthalmic device of claim 1, wherein the second optical characteristic comprises one or more of a tinting, a filter, a density filter, or a neutral density filter.

8. The ophthalmic device of claim 1, wherein an optical density of the second optical characteristic of the first lens is between about 0.05 and about 0.3.

9. A method comprising:
outputting a first representation of a moving object to a first eye of a user;
outputting a second representation of the moving object to a second eye of the user, wherein the second representation is viewed by the second eye via a lens that modifies a focal point of the second eye to be different than a focal point of the first eye;
receiving data indicative of an adjustment to a characteristic of one or more of the first representation or the second representation;
determining, based on the data indicative of the adjustment, a lens characteristic associated with reducing a misperception of distance of the moving object; and
outputting data indicative of the lens characteristic.

10. The method of claim 9, wherein receiving data indicative of the adjustment comprises receiving data indicative of an adjustment that prevents the user from having a perception of depth in the moving object.

11. The method of claim 9, wherein determining the lens characteristic comprises determining one or more of an optical density, a tinting, a density filter, a virtual filter, a virtual density filter, or a neutral density filter.

12. The method of claim 9, wherein the lens characteristic is associated with eliminating the misperception of distance of the moving object.

13. The method of claim 9, wherein the first representation comprises a first monocular image of a binocular display and the second representation comprises a second monocular image of the binocular display.

14. The method of claim 9, further comprising updating, based on the data indicative of the adjustment, one or more of the first representation or the second representation to reduce the misperception of distance of the moving object.

15. (canceled)

16. The method of claim 9, wherein the first representation is viewed by the first eye via an additional lens.

17. The method of claim 16, wherein the additional lens modifies a distance of focal point of the first eye such that the distance of the focal point of the first eye as modified by the additional lens is different from the distance of the focal point of the second eye as modified by the lens.

18. (canceled)

19. The method of claim 9, wherein the lens characteristic comprises one or more of an optical characteristic of the lens that modifies the focal point of the second eye or an optical characteristic of an additional lens for the first eye.

20-21. (canceled)

22. An ophthalmic system, comprising:
a first lens having a first optical characteristic that modifies a distance of a focal point of a first eye; and
a second lens having a second optical characteristic that modifies a distance of a focal point of a second eye, wherein the distance of the focal point of the first eye modified by the first lens is different than the distance of the focal point of the second eye modified by the second lens, and wherein the second lens has a third optical characteristic to reduce a misperception of a distance of a moving object.

23. The ophthalmic system of claim 22, wherein the first lens does not have the third optical characteristic or has less of the third optical characteristic than the second lens.

24. The ophthalmic system of claim 22, wherein one or more of the first lens or the second lens comprises one or more of a contact lens, an intraocular lens, an ocular implant, an ocular inlay, an ocular onlay, a lens mounted on a wearable frame, or a virtual lens formed by addition, removal, or reshaping of ocular media.

25. The ophthalmic system of claim 22, wherein the first lens corrects refractive errors of the first eye and the second lens corrects refractive errors of the second eye.

26-28. (canceled)
